(19) United States
(12) Patent Application Publication
(10) Pub. No.: US 2012/0035105 A1
(43) Pub. Date: Feb. 9, 2012

(54) Insulin Therapies for the Treatment of Diabetes, Diabetes Related Ailments, and/or Diseases or Conditions Other Than Diabetes or Diabetes Related Ailments

(75) Inventors: W. Blair Geho, Wooster, OH (US); John R. Lau, Howard, OH (US)

(73) Assignee: SDG, Inc.

(21) Appl. No.: 13/143,435

(22) PCT Filed: Jan. 8, 2010
(86) PCT No.: PCT/US10/20467

§ 371 Date: Sep. 26, 2011

Related U.S. Application Data
(60) Provisional application No. 61/143,512, filed on Jan. 9, 2009.
(60) Provisional application No. 61/250,309, filed on Oct. 9, 2009.

(51) Int. Cl. A61K 38/28; A61P 3/10

(52) U.S. Cl. 514/6.5

(57) ABSTRACT

The present invention includes a pharmaceutical composition comprising HDV insulin or oral HDV insulin, and one or more additional therapeutic agents useful for the treatment of diabetes and diabetes related ailments. The present invention also includes a method of making the pharmaceutical inventions of the application. The present invention further includes methods of treating diabetes and/or diabetes related ailments comprising administering a pharmaceutical composition of the invention to a patient in need thereof. The present invention also includes methods of treating diabetes related ailments comprising administering a pharmaceutical formulation of HDV insulin or a pharmaceutical formulation of oral HDV insulin.

### BACKGROUND OF THE INVENTION

The use of insulin to treat diabetes, diabetes related ailments, and diseases or conditions other than diabetes or diabetes related ailments is often complicated by the fact that, in addition to insulin, patients may need to be dosed with one or more additional therapeutic agents other than insulin. This requires multiple painful injections of insulin and a separate drug maintenance schedule for the one or more additional therapeutic agents other than insulin. As a result, what is needed for the treatment of diabetes, diabetes related ailments, and diseases or conditions other than diabetes or diabetes related ailments, is a combination therapy that obviates the need for separate drug maintenance schedules.

### BRIEF SUMMARY OF THE INVENTION

The present invention includes a pharmaceutical composition comprising HDV insulin and one or more additional therapeutic agents not associated with the HDV insulin. The present invention also includes a pharmaceutical composition comprising oral HDV insulin and one or more additional therapeutic agents not associated with oral HDV insulin.

The present invention also includes methods of treating diabetes, diabetes related ailments, and diseases or conditions other than diabetes and diabetes related ailments, comprising administering a pharmaceutical composition comprising HDV insulin and one or more additional therapeutic agents not associated with the HDV insulin. The present invention also includes methods of treating diabetes, diabetes related ailments, and diseases or conditions other than diabetes and diabetes related ailments, comprising administering a pharmaceutical composition comprising oral HDV insulin and one or more additional therapeutic agents not associated with oral HDV insulin.

The present invention also includes methods of treating diabetes related ailments and diseases or conditions other than diabetes, comprising administering HDV insulin or oral HDV insulin in the absence of any additional therapeutic agents.

The present invention further includes a method of treating diabetes, diabetes related ailments, and diseases or conditions other than diabetes or diabetes related ailments, comprising administering HDV insulin or oral HDV insulin and co-administering one or more additional therapeutic agents.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a schematic representation of HDV

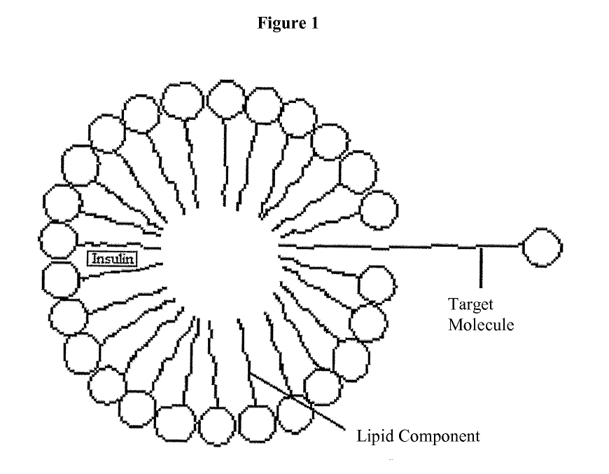

FIG. 2 is a schematic representation of oral HDV insulin.

FIG. 3 is a graph of the size distribution of the constituent members that comprise the oral HDV construct.

### DETAILED DESCRIPTION OF THE INVENTION

The present invention includes a pharmaceutical composition comprising HDV insulin and one or more additional therapeutic agents not associated with the HDV insulin. The present invention also includes a pharmaceutical composition comprising oral HDV insulin and one or more additional therapeutic agents not associated with oral HDV insulin.

The present invention also includes methods of treating diabetes, diabetes related ailments, and diseases or conditions other than diabetes and diabetes related ailments, comprising administering a pharmaceutical composition comprising HDV insulin and one or more additional therapeutic agents not associated with the HDV insulin. The present invention also includes methods of treating diabetes, diabetes related ailments, and diseases or conditions other than diabetes and diabetes related ailments, comprising administering a pharmaceutical composition comprising oral HDV insulin and one or more additional therapeutic agents not associated with oral HDV insulin.

The present invention also includes methods of treating diabetes related ailments and diseases or conditions other than diabetes, comprising administering HDV insulin or oral HDV insulin in the absence of any additional therapeutic agents.

The present invention further includes a method of treating diabetes, diabetes related ailments; and diseases or conditions other than diabetes or diabetes related ailments, comprising administering HDV insulin or oral HDV insulin and co-administering one or more additional therapeutic agents.

### DEFINITIONS

Unless defined otherwise, all technical and scientific terms used herein generally have the same meaning as commonly understood by one of ordinary skill in the art to which the invention belongs. Generally, the nomenclature used herein and the laboratory procedures in organic chemistry and protein chemistry are those well known and commonly employed in the art.

The articles “a” and “an” are used herein to refer to one or to more than one (i.e. to at least one) of the grammatical object of the article. By way of example, “an element” means one element or more than one element.

As used herein, amino acids are represented by the full name thereof, by the three-letter code as well as the one-letter code corresponding thereto, as indicated in the following table:

| | 3 Letter | 1-Letter |
|---|---|---|
| Full Name | Code | Code |
| Alanine | Ala | A |
| Arginine | Arg | R |
| Asparagine | Asn | N |
| Aspartic Acid | Asp | D |
| Cysteine | Cys | C |
| Cystine | Cys-Cys | C-C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “lower”, when used in reference to a chemical structure, describes a group containing from 1 to 6 carbon atoms.

The term “alkyl”, by itself or as part of another substituent means, unless otherwise stated, a straight, branched or cyclic hydrocarbon having the number of carbon atoms designated (i.e. C₁-C₆ means one to six carbons). Examples include: methyl, ethyl, propyl, isopropyl, butyl, isobutyl, test-butyl, pentyl, neopentyl, hexyl, cyclohexyl and cyclopropylmethyl. Most preferred is (C₁-C₃)alkyl, particularly ethyl, methyl and isopropyl.

The term “alkylene”, by itself or as part of another substituent means, unless otherwise stated, a straight, branched or cyclic chain hydrocarbon having two substitution sites, e.g., methylene (—CH₂—), ethylene (—CH₂CH₂—), isopropylene (—C(CH₃)═CH—), etc.

The term “aryl”, employed alone or in combination with other terms, means, unless otherwise stated, a carbocyclic structure, with or without saturation, containing one or more rings (typically one, two or three rings) wherein said rings may be attached together in a pendant manner, such as a biphenyl, or may be fused, such as naphthalene. Examples include phenyl, anthracyl, and naphthyl. The structure may be optionally substituted with one or more substituents, independently selected from halogen; (C₁-C₆)alkyl; (C₁-C₆)alkenyl; (C₁-C₆)alkoxy; OH; NO₂; C≡N; C(═O)O(C₁-C₃)alkyl; (C₂-C₆)alkylene-OR²; phosphonato; NR²₂; NHC(═O)(C₁-C₆)alkyl; sulfamyl; carbamyl; OC(═O)(C₁-C₃)alkyl; O(C₂-C₆)alkylene-N((C₁-C₆)alkyl)₂; and (C₁-C₃)perfluoroalkyl.

The term “arylloweralkyl” means a functional group wherein an aryl group is attached to a lower alkylene group, e.g., —CH₂CH₂-phenyl.

The term “alkoxy” employed alone or in combination with other terms means, unless otherwise stated, an alkyl group or an alkyl group containing a substituent such as a hydroxyl group, having the designated number of carbon atoms connected to the rest of the molecule via an oxygen atom, such as, for example, —OCH(OH)—, —OCH₂OH, methoxy (—OCH₃), ethoxy (—OCH₂CH₃), 1-propoxy (—OCH₂CH₂CH₃), 2-propoxy (isopropoxy), butoxy (—OCH₂CH₂CH₂CH₃), pentoxy (—OCH₂CH₂CH₂CH₂CH₃), and the higher homologs and isomers.

The term “acyl” means a functional group of the general formula —C(═O)—R, wherein —R is hydrogen, alkyl, amino or alkoxy. Examples include acetyl (—C(═O)CH₃), propionyl (—C(═O)CH₂CH₃), benzoyl (—C(═O)C₆H₅), phenylacetyl (C(═O)CH₂C₆H₅), carboethoxy (—CO₂CH₂CH₃), and dimethylcarbamoyl (C(═O)N(CH₃)₂).

The terms “halo” or “halogen” by themselves or as part of another substituent mean, unless otherwise stated, a fluorine, chlorine, bromine, or iodine atom.

The term “heterocycle” or “heterocyclyl” or “heterocyclic” by itself or as part of another substituent means, unless otherwise stated, a saturated or unsaturated, stable, mono or multicyclic ring system comprising carbon atoms and at least one heteroatom selected from the group comprising N, O, and S, and wherein the nitrogen and sulfur heteroatoms may be optionally oxidized, and the nitrogen atom may be optionally quaternized. Examples include pyridine, pyrrole, imidazole, benzimidazole, phthalein, pyridenyl, pyranyl, furanyl, thiazole, thiophene, oxazole, pyrazole, 3-pyrroline, pyrrolidene, pyrimidine, purine, quinoline, isoquinoline, carbazole, etc. Where substitution will result in a stable compounds, the structure may be optionally substituted with one or more substituents, independently selected from halogen; (C₁-C₆)alkyl; (C₁-C₆)alkenyl; (C₁-C₆)alkoxy; OH; NO₂; C≡N; C(═O)O(C₁-C₃)alkyl; (C₂-C₆)alkylene-OR²; phosphonato; NR²₂; NHC(═O)(C₁-C₆)alkyl; sulfamyl; carbamyl; OC(═O)(C₁-C₃)alkyl; O(C₂-C₆)alkylene-N((C₁-C₆)alkyl)₂; and (C₁-C₃)perfluoroalkyl.

The term “amphipathic lipid” means a lipid molecule having a polar end and a non-polar end.

A “complexing agent” is a compound capable of forming a water insoluble coordination complex with a metal, e.g. a salt of chromium, zirconium, etc., that is substantially insoluble in water and soluble in organic solvents.

“Aqueous media” means media comprising water or media comprising water containing at least one buffer or salt.

The terms “associated,” or “associated with” when used in reference to a composition or constituent of a composition of this invention, means that the referenced material is incorporated (or intercalated) into, or on the surface of, or within a composition or a constituent of a composition of the present invention.

The term “insulin” refers to natural or recombinant forms of insulin, synthetic insulin, and derivatives of the aforementioned insulins. Examples of insulin include, but are not limited to insulin lispro, insulin aspart, regular insulin, insulin glargine, insulin zinc, human insulin zinc extended, isophane insulin, human buffered regular insulin, insulin glulisine, recombinant human regular insulin, ultralente insulin, humulin, NPH insulin, Levemir, Novolog, and recombinant human insulin isophane. Also included are animal insulins, such as bovine or porcine insulin.

The terms “glargine” and “glargine insulin” both refer to a recombinant human insulin analog which differs from human insulin in that the amino acid asparagine at position A21 is replaced by glycine and two arginines are added to the C-terminus of the B-chain. Chemically, it is 21A-Gly-30Ba-L-Arg-30Bb-L-Arg-human insulin and has the empirical formula C₂₆₇H₄₀₄N₇₂O₇₈S₆ and a molecular weight of 6063.

The term “recombinant human insulin isophane” refers to a human insulin that has been treated with protamine.

The phrase “HDV insulin” as used herein refers to insulin associated with a composition that enables targeted delivery of insulin to hepatocytes.

The phrase “oral HDV insulin” as used herein refers to insulin associated with an orally bioavailable lipid construct that permits oral delivery of insulin. Like regular HDV insulin, oral HDV insulin enables targeted delivery of insulin to hepatocytes.

The term “bioavailability” refers to a measurement of the rate and extent that insulin and/or other therapeutic agent reaches the systemic circulation and is available at its site of action.

As used herein, “co-administration” or “co-administering” or “combination therapy” as well as variations thereof, mean administering HDV insulin or oral HDV insulin before, during, or after the administration of one or more additional therapeutic agents wherein the one or more additional therapeutic agents is not associated with HDV insulin or oral HDV insulin. Co-administration may take place via the same or different routes of administration. Co-administration may be concurrent, sequential, or spaced at specific time intervals. Co-administration need not, however, take place within a set time period. As such, and by way of example only, administration of HDV insulin at any time before or after the administration of one or more additional therapeutic agents constitutes co-administration so long as either HDV insulin or the one or more additional therapeutics (whichever is administered first) is still present in the patient at the time of co-administration. In certain embodiments, though, the first administered compound need not be present in the patient at the time of co-administration.

As used herein, “to treat”, “treatment”, “treating”, as well as variations thereof, mean reducing the frequency with which symptoms of a disease, disorder, or adverse condition, and the like, are experienced by a patient. Treating a patient may further include slowing or preventing the onset, development, or progression of a particular disease or disorder. Treating may further include curing a patient.

As used herein, the term “pharmaceutically acceptable carrier” means a chemical composition with which the active ingredient may be combined and which, following the combination, can be used to administer the active ingredient to a subject.

The term “lipid” or “lipids” means an organic compound characterized by its preference for non-polar aprotic organic solvents. A lipid may or may not possess an alkyl tail. Lipids according to the present invention include, but are not limited to, the class of compounds known in the art as phospholipids, cholesterols, and dialkyl phosphates.

As used herein, “cholesterol” means the compound and all derivatives and analogs of the compound:

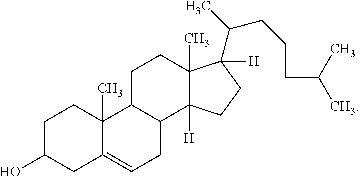

As used herein, “particle” comprises an agglomeration of multiple units of one or more lipids.

As used herein, “diabetes related ailments” include, but are not limited to, diseases or conditions including obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia or decreased mental acuity, as well as any other disease, symptom, or condition, related to, caused by, or otherwise associated with the diabetic condition.

As used herein, “diseases or conditions other than diabetes and diabetes related ailments” include cancer, reducing peripheral insulin levels, weight management, weight loss, and administration of insulin before, during, or after surgery as an anti-stress metabolic enhancement agent.

As used herein “cardiovascular disease” includes, but is not limited to, atherosclerosis, hyperlipidaemias, such as elevated LDL or triglycerides, angina pectoris, hypertension, or cardiac risk.

The term “therapeutic agent” as used herein refers to the non-insulin class of compounds useful for the treatment of diabetes, diabetes related ailments, and/or affecting diseases or conditions other than diabetes or diabetes related ailments. Examples of therapeutic agents include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, Serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, Monoclonal antibodies (“MAbs”) to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

As used herein “α-glucosidase inhibitor,” includes, but is not limited to, acarbose, miglitol, and voglibose.

As used herein “lipase inhibitor,” includes, but is not limited to, orlistat.

As used herein “sulfonyl urea” includes, but is not limited to, acetohexamide, chlorpropamide, tolbutamide, tolazamide, gliclazide, glyburide, glibenclamide, glipizide, glimepiride, and gliquidone.

As used herein “meglitinide” includes, but is not limited to, mitiglinide, nateglinide, and repaglinide.

As used herein “biguanide” includes, but is not limited to, metformin, phenformin, and buformin.

As used herein “thiazolidinedione” includes, but is not limited to, rosiglitazone, pioglitazone, troglitazone, and tesaglitazar.

As used herein “incretin mimetic” includes, but is not limited to, exenatide, and liraglutide.

As used herein “ ”GLP-1 receptor agonist” includes, but is not limited to, GLP-1, As used herein “DPP-IV inhibitor” includes, but is not limited to, sitagliptin, a combination of sitagliptin and metformin, vildagliptin, and a combination of vildagliptin and metformin, alogliptin, a combination of alogliptin and metform, saxagliptin, ABT-279 (Abbott Laboratories), AMG222 (Amgen), KRP-104 (ActivX Biosciences), MP-513 (Mitsubishi Pharma), linagliptin, saxagliptin, PF-734200 (Pfizer), PHX-1149 (Phenomix/Forest Laboratories, R1579 (Roche), SYR-472 (Takeda Pharmaceuticals), and TA-6666 (Mitsubishi Pharma).

As used herein “niacin,” includes but is not limited to, immediate and controlled release formulations of niacin, Niacin also includes metabolites of niacin which may be synthesized and dosed independently of the parent niacin molecule.

As used herein “fibrate” includes, but is not limited to, fenofibrate, bezafibrate, and gernfibrozil.

As used herein “bile acid sequestrant” includes, but is not limited to, colesevelam and cholestyramine.

As used herein “cholesterol absorption inhibitor” includes, but is not limited to, ezetimibe, FM-VP4, AEGR-733, implitapide and JTT-130.

As used herein “omega-3 acid ethyl esters” includes, but is not limited to, Omacor™, Esapent™, Seacor™, and Maxepa™.

As used herein “secretory phospholipase A2 inhibitor” or“sPLA2 inhibitor,” includes, but is not limited to, S-5920, LY315920, and A-002. These experimental drugs are available from Anthera Pharmaceuticals, Inc.

As used herein “oligonucleotide-based apolipoprotein B inhibitor,” (or “ApoB inhibitor”) includes, but is not limited to, mipomersen sodium.

As used herein “statin” includes, but is not limited to, mevastatin, lovastatin, simvastatin, pravastatin, fluvastatin, pitavastatin, atorvastatin, cerivastatin, and rosuvastatin.

As used herein “squalene synthase inhibitor” includes, but is not limited to, lapaquistat.

As used herein “fixed dose combination statin therapy” includes, but is not limited to, Vytorin™ (simvastatin and ezetimibe), Caduet™ (atorvastatin and amlodipine), and Advicorml (lovastatin and nicotinic acid).

As used herein “angiotensin II receptor antagonist” includes, but is not limited to, valsartan, losartan, irbesartan, candesartan celexetil, and olmesartan. Angiotensin II Receptor Antagonists also include combination therapies such as combinations of losartan and hydrochlorothiazide, valsartan and hydrochlorothiazide.

As used herein “ACE inhibitors” includes, but is not limited to, benazepril, captopril, lisinopril, ramipril, and enalapril. ACE inhibitors also include combination therapies such as combinations of lisinopril and hydrochlorothiazide, and a combination of benazepril and amlodipine.

As used herein “antidepressant” includes, but is not limited to, amitriptyline, imipramine, desipramine, duloxetine, venlafaxin, bupropion, paroxetine, citalopram, dapoxetine, escitalopram, fluoxetinc, fluvoxamine, paroxetine, sertraline, and zimelidine.

As used herein “anticonvulsant” includes, but is not limited to, pregabalin, gabapentin, carbamazepine, lamotrigine, and topiramate.

As used herein “opioid” refers to both actual opioids as well as opioid-like drugs. Examples include, but are not limited to, morphine, codeine, thebaine, hydromorphone, hydrocodone, oxycodone, oxymorphone, desomorphine, nicomorphine, dipropanoylmorphine, benzylmorphine, ethylmorphine, fentanyl, pethidine, methadone, tramadol and propoxyphene.

As used herein “aldose reductase inhibitor” includes, but is not limited to, epalrestat and ranircstat.

As used herein “pancreatic lipase inhibitor” includes, but is not limited to orlistat and cetilistat.

As used herein “serotonin-norepinephrine reuptake inhibitor” includes, but is not limited to, sibutramine.

As used herein “cannabinoid receptor antagonist” (or “CBI receptor antagonist”) includes, but is not limited to, rimonabant and MK-0364.

As used herein “anti-obesity combination therapy” includes, but is not limited to, a combination of topiramate and phentermine, a combination of bupropion and zonisamide, a combination of bupropion and naltrexone, a combination of phentermine and fluoxetine, a combination of phentermine and sertraline, a combination of phentermine and citalopram, a combination of phentermine and escitalopram, and a combination of phentermine and trazadone.

As used herein “erectile dysfunction medication” includes, but is not limited to alprostadil, tadalafil, vardenafil, and sildenafil.

As used herein, “alpha-1-adrenergic receptor blockers” include, but are not limited to, doxazosin, prazosin, trimazosin, tamsulosin, alfuzosin, terazosin, phenoxybenzamine, and phentolamine.

As used herein, “5-alpha reductase inhibitors” include, but are not limited to finasteride, dutasteride, isotretinoin, and FCE 28260.

As used herein “fish oil” includes, but is not limited to, omega-3-acid ethyl esters. Examples of omega-3-acide ethyl esters include eicosapentaenoic acid (“EPA”) and docosahexaenoic acid (“DHA”), as well as combinations thereof.

As used herein “plant sterols and stanols” include, but are not limited to, β-sitosterol, β-sitostanol, campesterol, and sigmasterol, as well as combinations thereof.

As used herein “immunosuppressors” include, but are not limited to, cyclosporine, prednisone, a combination of prednisone and azathioprine, azathioprine, rapamycine, anti-CD 3 mAb, IL10, a combination of sirolimus and tacrolimus, vitamin D, a combination of cyclophosphamide and antithymocyte globulin, mycophenoalte mofetil, anti-IL2 receptor Ab, anti-CD20 Ab, anti-thymocyte globulin, somatostatin, and diazoxide.

As used herein “11βHSD1 inhibitors” include, but are not limited to 11βHSD inhibitor (Bristol-Myers Squibb), AMG 221 (Amgen), HSD016 (Wyeth Pharmaceuticals), INCB-13739 (Incyte), INCB-20817 (Incyte), and JTT-654 (Akros Pharma).

As used herein adenosine A1 receptor agonists include, but are not limited to, CVT-3619 (CV Therapeutics).

As used herein “anti-inflammatory agents” include, but are not limited to, lisofylline (DiaKine Therapeutics), HE3286 (Hollis-Eden), VGX-1027 (VGX Pharmaceuticals), and succinobucol (AtheroGenics).

As used herein “artificial sweeteners” include, but are not limited to, tagatose. As used herein “bile acid receptor agonists” include, but are not limited to, 756050 (GSK), INT-747 (Intercept Pharmaceuticals), INT-767 (Intercept Pharmaceuticals), and INT-777 (Intercept Pharmaceuticals).

As used herein “CCK receptor antagonists” include, but are not limited to, CE-326597 (Pfizer).

As used herein “CCR2Antagonists” include, but are not limited to, CCR2 antagonist (BMS).

As used herein “diacylglycerol O-acyltransferase homolog 1 (DGAT-1) inhibitors” include, but are not limited to, PF-4620110.

As used herein “dopamine receptor agonists” include, but are not limited to bromocriptine.

As used herein “dual-acting peptide-GLP-1 and glucagons receptor agonists” include, but are not limited to, BAY 73-7977 (Bayer).

As used herein “fructose 1,6 bisphosphatase inhibitors” include, but are not limited to, MB07803 (Metabasis Therapeutics).

As used herein “gastrin-releasing peptide (GRP) receptor agonists” include, but are not limited to 1292263 (GSK).

As used herein “GLP-1 analogs” include, but are not limited to, R1583 (Roche)

As used herein “glucagons receptors-antisense” include, but are not limited to, OMJP-GCGR (ISIS 325568) (Isis Pharmaceuticals).

As used herein “glucokinase activators” include, but are not limited to LY2599506 (Eli Lilly), NN9101 (Novo Nordisk), R1511 (Roche), TTP355 (TransTech Pharma), and MK-0941 (Merck).

As used herein “glucose-dependent insulinotropic receptor (GDIR/GPR119) agonists” include, but are not limited to MBX-298 (Metabolex) and PSN821 (OSI Pharmaceuticals).

As used herein “glutamic acid decarboxylases” include, but are not limited to, DIAMYD (Diamyd Medical).

As used herein “HM74a agonists” include, but are not limited to INCB-19602 (Incyte).

As used herein “HSP60 peptides” include, but are not limited to DiaPep277®.

As used herein “insulin-derived peptides” include, but are not limited to NBT-6024 (Neurocrine Biosciences).

As used herein “longer acting human GLP-1 analogues” include but are not limited to NN9535 (Novo Nordisk).

As used herein “MAbs to CD3” include, but are not limited to otelixizuman (Tolerx/GSK) and teplizumab (Eli Lilly).

As used herein “MAbs to glucagon receptors” include, but are not limited to AMG 477 (Amgen).

As used herein “MAbs to IL-1” include, but are not limited to AMG 108 (Amgen).

As used herein “MAbs to IL-1β” include, but are not limited to canakinumab (Novartis Pharmaceuticals) and XOMA052 (XOMA).

As used herein “permeability inhibitors” include, but are not limited to larazotide (Alba Therapeutics).

As used herein “plasmid encoding proinsulins” include but are not limited to BHT-3021 (Bayhill Therapeutics).

As used herein “poly(ADP-ribose) polymerase inhibitors” include, but are not limited to BGP-15 (N-Gene Research).

As used herein “PPAR agonists” include, but are not limited to, SAR351034 (Sanofi-Aventis).

As used herein “PPAR alpha activators” include, but are not limited to, K-111 (Kowa Pharmaceuticals).

As used herein “PPAR gamma modulators” include, but are not limited to, 376501 (GSK), balaglitazone (Dr. Reddy's Laboratories), INT-131 (InteKrin Therapeutics), MBX-102 (JM-39659100) (Johnson & Johnson/Metabolex), MBX-2044 (Johnson & Johnson/Metabolex), Mitoglitazone™ (Metabolic Solutions), netoglitazone (perlegen Sciences), rivoglitazone (Daiichi Sankyo), and MK-0893 (Merck).

As used herein “PPAR pan agonists” include, but are not limited to, 625019 (GSK) and sodelglitazar (GSK).

As used herein “PPARα/γ modulators” include, but are not limited to, DSP-8658 (Dainippon Sumitomo), ONO-5129 (Ono Pharma), and R1439/aleglitazar (Roche).

As used herein “protein tyrosine phosphatase 1B inhibitors” include, but are not limited to, ISIS113715 (Isis Pharmaceuticals) and trodusquemine (Genaera).

As used herein “SGLT1 inhibitors” include, but are not limited to, 1614235 (GSK),

As used herein “SGLT2 inhibitors” include, but are not limited to, BI-10773 (Boehringer Ingelheim), BI-44847 (Boehringer Ingelheim), Dapagliflozin (AstraZeneca), ISIS-SGLT2rx (Isis Pharmaceuticals), LX-4211 (Lexicon Pharmaceuticals), R7201 (Roche), remogliflozin (GSK), TA-7284 (JNJ-28431754) (Johnson & Johnson/Mitsubishi), YM543 (Astellas Pharma US), and ASP 1941 (Astellas Pharma US).

As used herein “SIAC (soluble insulin analogue combinations)” include, but are not limited to NN5401 (Novo Nordisk).

As used herein, “soluble insulin basal analogues” include, but are not limited to, NN1250 (Novo Nordisk).

As used herein, sirtuin (SIRT1) activators include, but are not limited to, 2245840 (GSK), 184072 (GSK), SRT501 (resveratrol) (Sirtris Pharmaceuticals), and SRT2104 (Sirtris Pharmaceuticals).

As used herein “sodium channel blockers” include, but are not limited to, pyrazinoylguanidine (SuperGen).

As used herein “other non-insulin compounds” include, but are not limited to DC9703 (Obio Pharmaceuticals), EX-1000 (Novo Nordisk), MK-1006 (Merck), MK-4074 (Merck), MK-8245 (Merck), NP-500 (Napo Pharmaceuticals), PF-4325667 (Pfizer), PPM-201 (Wyeth Pharmaceuticals), PPM-202 (Wyeth Pharmaceuticals), R4929 (Roche), R7089 (Roche), R7234 (Roche), RO-438857 (Roche), RO-4998452 (Roche), TAK-875 (Takeda Pharmaceuticals), and CRx-401 (CombinatoRx).

### HDV Insulin

HDV insulin is comprised of insulin associated with a lipid construct. The lipid construct is comprised of one or more lipid components selected from the group consisting of 1,2-distearoyl-sn-glycero-3-phosphocholine, 1,2-dipalmitoyl-sn-glycero-3-phosphocholine, 1,2-dimyristoyl-sn-glycero-3-phosphocholine, cholesterol, cholesterol oleate, dihexadecyl phosphate, 1,2-distearoyl-sn-glycero-3-phosphate, 1,2-dipaltnitoyl-sn-glycero-3-phosphate, 1,2-dimyristoyl-sn-glycero-3-phosphate, 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt), triethylammonium 2,3-diacetoxypropyl 2-(5-((3aS,6aR)-2-oxohexahydro-1H-thieno[3,4-d]imidazol-4-yl)pentanamido)ethyl phosphate, and derivatives thereof. Representative structures are presented in Table 1.

TABLE 1

| Common Name | Chemical Name | Structure |
|---|---|---|
| 1,2-distearoyl- sn-glycero-3- phosphocholine | 2,3- bis(stearoyloxy)propyl 2-(trimethylammonio) ethyl phosphate | 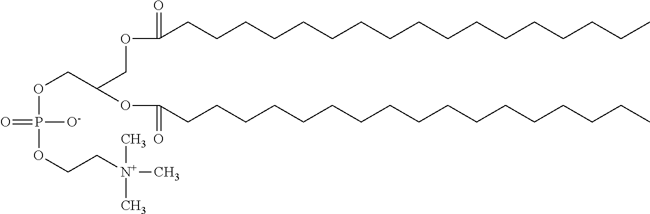 |
| 1,2-dipalmitoyl- sn-glycero-3- phosphocholine | 2,3- bis(palmitoyloxy)propyl 2-(trimethylammonio) ethyl phosphate | 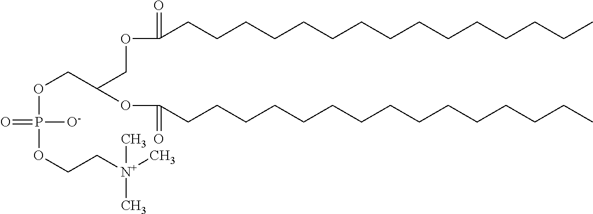 |
| 1,2-dimyristoyl- sn-glycero-3- phosphocholine | 2,3-bis (tetradecanoyloxy) propyl 2- (trimethylammonio) ethyl phosphate | 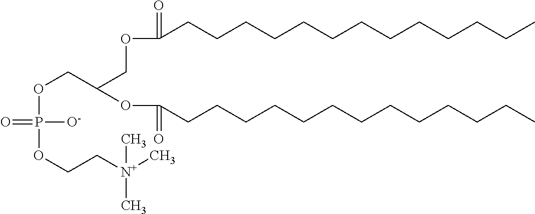 |
| Cholesterol | 10,13-dimethyl-17- (6-methylheptan-2-yl)- 2,3,4,7,8,9,10,11,12,13, 14,15,16,17- tetradecahydro-1H- cyclopenta[a]phenanthren- 3-ol | 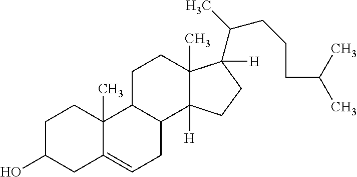 |

Preferably, the lipid construct components are 1,2-distearoyl-sn-glycero-3-phosphocholine, cholesterol, and dihexadecyl phosphate.

The lipid construct further comprises at least one targeting agent. Targeting agents are discussed, at length, below. Preferably, however, the targeting agent is biotin DHPE, biotin-X-DHPE, or poly[Cr-bis(N-2,6-diisopropylphenylcarbamoylmethyl iminodiacetic acid)].

In one embodiment, the lipid construct was prepared by mixing 1,2-distearoyl-sn-glycero-3-phosphocholine, cholesterol, dihexadecyl phosphate, and targeting agent at about 71.2 wt %, 9.4 wt %, 18.2 wt %, and 1.2 wt %, respectively.

In another embodiment, the lipid construct may also include at least one diagnostic agent in combination with or in place of a targeting agent. Examples of diagnostic agents include diagnostic contrast agents such as, but not limited to, gold and a gadolinium. Other diagnostic agents include radioactive materials such as radioactive isotopes of common atoms including, but not limited to, ¹³C, ⁶⁸Ge, ¹⁸F, and ¹²⁵I. These contrast and radioactive agents are preferably covalently attached to a lipid component and/or targeting agent via known techniques in synthetic organic chemistry. Alternatively, and where chemically appropriate, the diagnostic agent may be bound to a ligand such as DADA (2′-deoxyadenosine), which is itself covalently attached to a lipid component or targeting agent via known techniques in synthetic organic chemistry.

### Targeting Agents

Targeting agents target insulin to a specific cellular or extracellular receptor. In one embodiment, a targeting agent facilitates delivery of insulin to the liver to control post-prandial glycogen storage and encompasses a class of molecules referred to as “hepatocyte target molecule” (HTM). HTM examples include, but are not limited to, biotin derived targeting agents such as 1,2-dipalmitoyl-sn-glycero-3-phosphoethanolamine-N-(biotinyl) (also referred to as biotin DHPE) and metal derived targeting agents such as poly[Cr-bis(N-2,6-diisopropylphenylcarbamoylmethyl iminodiacetic acid)]. Metal-derived targeting agents and biotin derived targeting agents are discussed below and are fully described in U.S. Pat. Nos. 7,169,410 and 4,603,044; PCT application PCT/US06/19119; and U.S. patent application Ser. Nos. 11/384,728, and 11/384,659. Additional examples of biotin-derived targeting agents are disclosed in Table 2.

When the targeting agent comprises biotin, iminobiotin, carboxybiotin, biocytin, or iminobiocytin, the biotin, iminobiotin, carboxybiotin, biocytin, or iminobiocytin molecules may be bound via an amide bond to the nitrogen of a phospholipid molecule such as 1,2-dipalmitoyl-sn-glycero-3-phosphoethanolamine. The compounds may likewise be bound to a molecule such as cholesterol through an ester linkage. In the case of biocytin and iminobiocytin, the compounds may be bound to benzoyl thioacetyl triglycine via an amide bond between the terminal nitrogen of iminiobiocytin and the terminal carbonyl of benzoyl thioacetyl triglycine. Alternative bond connectivities to those described above are possible and considered to be within the scope of the present invention.

TABLE 2

| | | |
|---|---|---|
| 1 | N-hydroxysuccinimide (NHS) biotin 2,5-dioxopyrrolidin-1- yl 5-((3aS,6aR)-2- oxohexahydro-1H- thieno[3,4-d]imidazol- 4-yl) pentanoate | 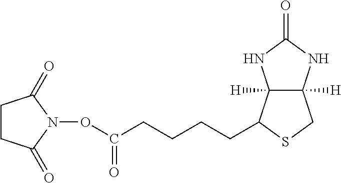 |
| 2 | sulfo-NHS-biotin sodium 2,5-dioxo-3- (trioxidanylthio) pyrrolidin-1-yl 5- ((3aS,6aR)-2- oxohexahydro-1H- thieno[3,4-d]imidazol- 4-yl) pentanoate | 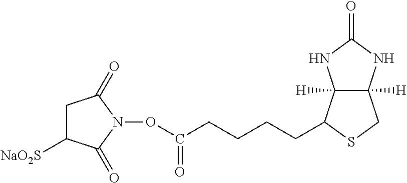 |
| 3 | N-hydroxysuccinimide long chain biotin 2,5-dioxopyrrolidin-1- yl 6-(5-((3aS,6aR)-2- oxohexahydro-1H- thieno[3,4-d]imidazol- 4-yl) pentanamido)hexanoate | 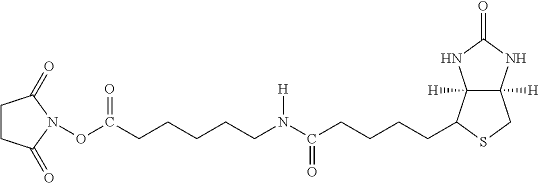 |
| 4 | sulfo-N- hydroxysuccinimide long chain biotin sodium 2,5-dioxo-3- (trioxidanylthio) pyrrolidin-1-yl 6-(5-((3aS,6aR)-2- oxohexahydro-1H- thieno[3,4-d]imidazol- 4-yl)pentanamido) hexanoate | 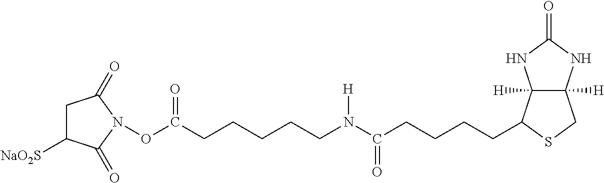 |
| 5 | D-biotin 5-((3aS,6aR)-2- oxohexahydro-1H- thieno[3,4-d]imidazol- 4-yl) pentanoic acid | 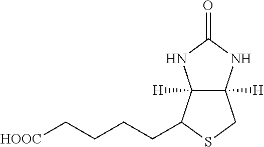 |
| 6 | Biocytin 2-amino-6- (5((3aS,6aR)-2- oxohexahydro-1H- thieno[3,4-d]imidazol- 4-yl) pentanamido) hexanoic acid | 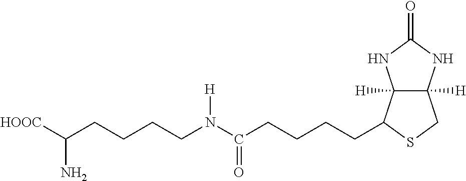 |
| 7 | sulfo-N- hydroxysuccinirnide-S- S-biotin sodium 2,5-dioxo-3- (trioxidanylthio) pyrrolidin-1-yl 3-((2-(4-((3aS,6aR)-2- oxohexahydro-1H- thieno[3,4-d]imidazol- 4-yl)butylamino) ethyl) disulfanyl)propanoate | 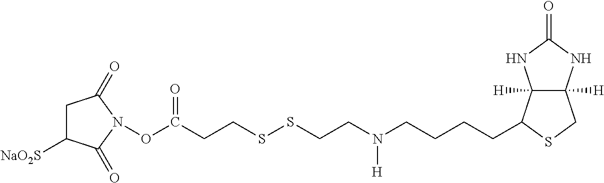 |
| 8 | biotin-BMCC 4-((2,5-dioxo-2,5- dihydro-1H-pyrrol-1- yl)methyl)-N-(4-(5- ((3aS,6aR)-2- oxohexahydro-1H- thieno[3,4-d]imidazol- 4-yl) pentanamido)butyl) cyclohexane- carboxamide | 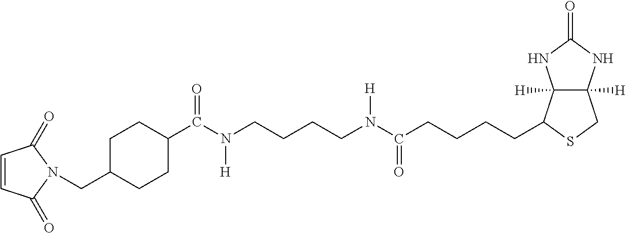 |
| 9 | biotin-HPDP 5-((3aS,6aR)-2- oxohexahydro-1H- thieno[3,4-d]imidazol- 4-yl)-N-(6-(3- (pyridin-2-yldisulfanyl) propanamido)hexyl) pentanamide | 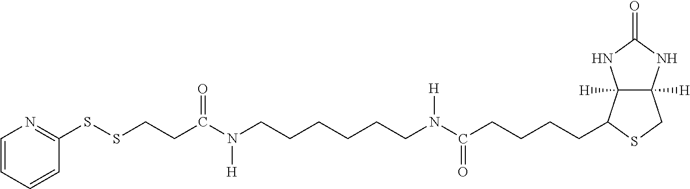 |
| 10 | iodoacetyl-LC-biotin N-(6-(2- iodoacetamido)hexyl)- 5-((3aS,6aR)-2- oxohexahydro- 1H-thieno[3,4- d]imidazol-4- yl)pentanamide | 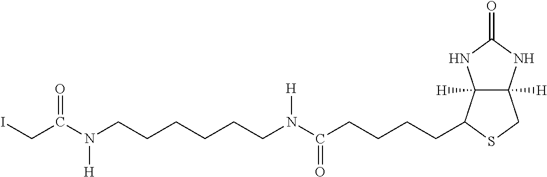 |
| 11 | biotin-hydrazide 5-((3aS,6aR)-2- oxohexahydro-1H- thieno[3,4-d]imidazol- 4-yl)pentanehydrazide | 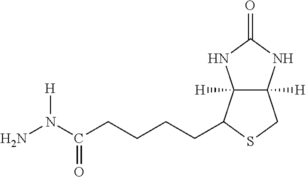 |
| 12 | biotin-LC-hydrazide N-(6-hydrazinyl-6- oxohexyl)-5-((3aS,6aR)- 2-oxohexahydro-1H- thieno[3,4-d]imidazol- 4-yl) pentanamide | 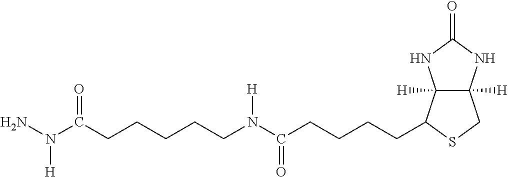 |
| 13 | biocytin hydrazide N-(5-amino-6- hydrazinyl-6-oxohexyl)- 5-((3aS,6aR)-2- oxohexahydro-1H- thieno[3,4-d]imidazol- 4-yl)pentanamide | 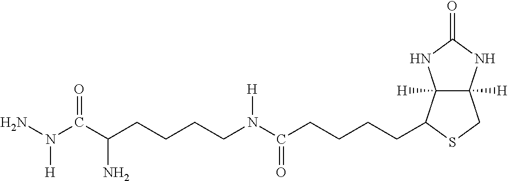 |
| 14 | biotin cadaverine N-(5-aminopentyl)- 5-((3aS,6aR)-2- oxohexahydro-1H- thieno[3,4-d]imidazol-4- yl)pentanamide | 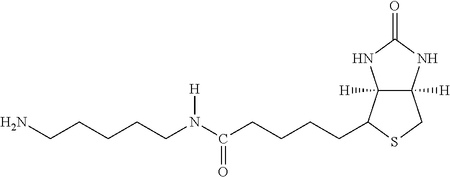 |
| 15 | Carboxybiotin (3aS,6aR)-4-(4- carboxybutyl)-2- oxohexahydro-1H- thieno[3,4-d]imidazole- 1-carboxylic acid | 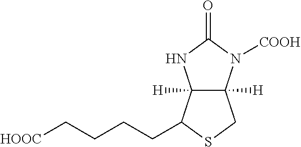 |
| 16 | Photobiotin N-(3-((3-(4-azido-2- nitrophenylamino) propyl)(methyl)amino) propyl)-5-((3aS,6aR)- 2-oxohexahydro-1H- thieno[3,4-d]imidazol- 4-yl)pentanamide | 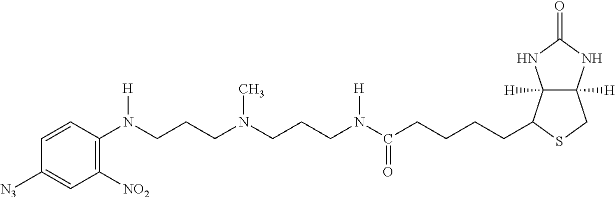 |
| 17 | ρ-aminobenzoyl biocytin trifluoroacetate 2-(4- aminobenzamido)-6- (5-((3aS,6aR)-2- oxohexahydro-1H- thieno[3,4-d]imidazol- 4-yl)pentanamido) hexanoic acid 2,2,2- trifluoroacetate | 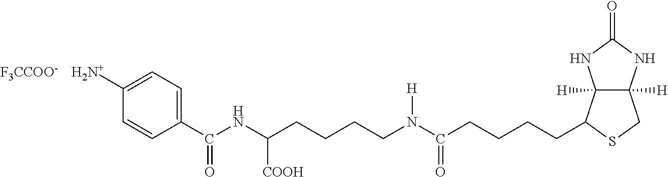 |
| 18 | ρ-diazobenzoyl biocytin 4-(1-carboxy-5-(5- ((3aS,6aR)-2- oxohexahydro-1H- thieno[3,4-d]imidazol- 4-yl)pentanamido) pentylcarbamoyl) benzenediazonium chloride | 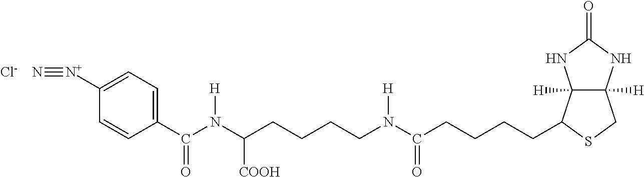 |
| 19 | biotin DHPE G⁺ = Li⁺, Na⁺, K⁺, (Et₃NH)⁺ 2,3-diacetoxypropyl 2-(5-((3aS,6aR)-2- oxohexahydro-1H- thieno[3,4-d]imidazol- 4-yl) pentanamido)ethyl phosphate | 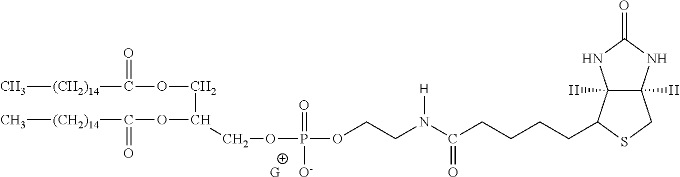 |
| 20 | biotin-X-DHPE G⁺ = Li⁺, Na⁺, K⁺, (Et₃NH)⁺ 2,3-diacetoxypropyl 2-(6-(5-((3aS,6aR)-2- oxohexahydro-1H- thieno[3,4-d]imidazol- 4-yl)pentanamido) hexananaido)ethyl phosphate | 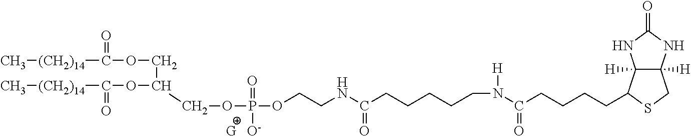 |
| 21 | 12-((biotinyl)amino) dodecanoic acid 12-(5-((3aS,6aR)-2- oxohexahydro-1H- thieno[3,4-d]imidazol- 4-yl) pentanamido) dodecanoic acid | 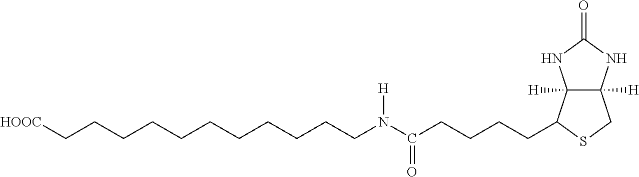 |
| 22 | 12-((biotinyl)amino) dodecanoic acid succinimidyl ester 2,5-dioxopyrrolidin-1- yl 12-(5-((3aS,6aR)-2- oxohexahydro-1H- thieno[3,4-d]imidazol- 4-yl) pentanamido) dodecanoate | 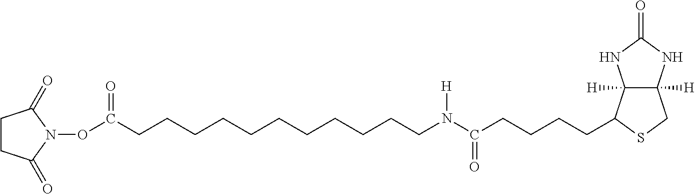 |
| 23 | S-biotinyl homocysteine 4-mercapto-2-(5- ((3aS,6aR)-2- oxohexahydro-1H- thieno[3,4-d]imidazol- 4-yl) pentanamido) butanoic acid | 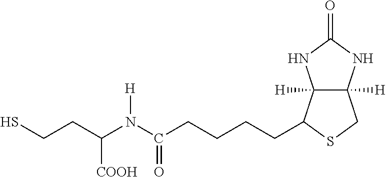 |
| 24 | biocytin-X 2-amino-6-(6-(5- ((3aS,6aR)-2- oxohexahydro-1H- thieno[3,4-d]imidazol- 4-yl)pentanamido) hexanamido)hexanoic acid | 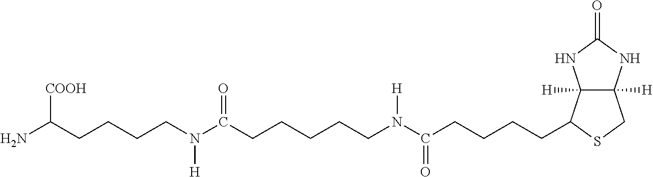 |
| 25 | biocytin x-hydrazide N-(5-amino-6- hydrazinyl-6-oxohexyl)- 6-(5-((3aS,6aR)-2- oxohexahydro-1H- thieno[3,4-d]imidazol- 4-yl)pentanamido) hexanamide | 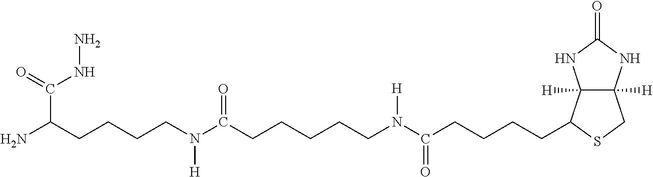 |
| 26 | Biotinethylenediamine N-(2-aminoethyl)-5- ((3aS,6aR)-2- oxohexahydro-1H- thieno[3,4-d]imidazol- 4-yl)pentanamide | 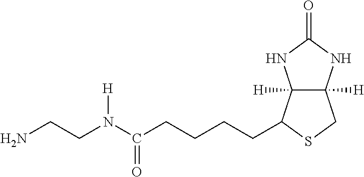 |
| 27 | biotin-X 6-(5-((3aS,6aR)-2- oxohexahydro-1H- thieno[3,4-d]imidazol- 4-yl)pentanamido) hexanoic acid | 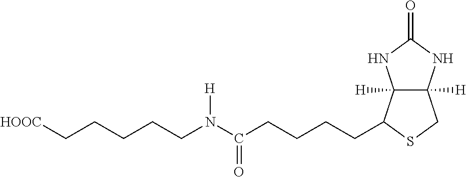 |
| 28 | biotin-X- ethylenediamine N-(2-aminoethyl)-6-(5- ((3aS,6aR)-2- oxohexahydro-1H- thieno[3,4-d]imidazol- 4-yl) pentanamido) hexanamide | 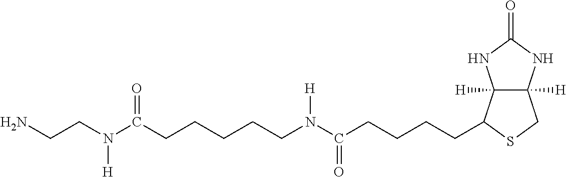 |
| 29 | biotin-composition hydrazide N-(6-hydrazinyl-6- oxohexyl)-6-(5- ((3aS,6aR)-2- oxohexahydro-1H- thieno[3,4-d]imidazol- 4-yl)pentanamido) hexanamide | 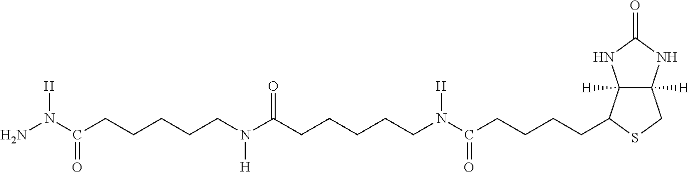 |
| 30 | biotin-composition-SE 2,5-dioxopyrrolidin-1- yl 6-(6-(5-((3aS,6aR)- 2-oxohexahydro-1H- thieno[3,4-d]imidazol- 4-yl) pentanamido) hexanamido) hexanoate | 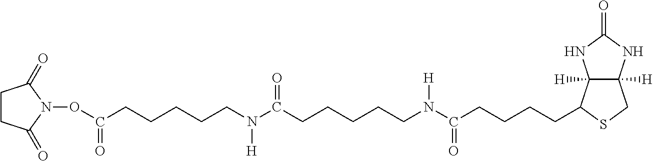 |
| 31 | biotin-composition, SSE sodium 2,5-dioxo-1- (6-(6-(5-((3aS,6aR)- 2-oxohexahydro-1H- thieno[3 ,4-d]imidazol- 4-yl)pentanamido) hexanamido) hexanoyloxy) pyrrolidine-3-sulfonate | 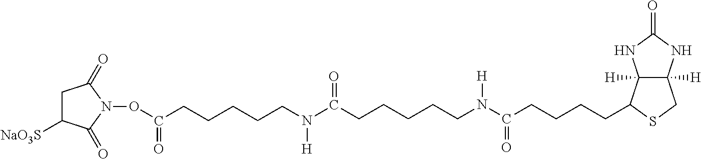 |
| 32 | biotin-X-cadaverine 5-(6-(5-((3aS,6aR)-2- oxohexahydro-1H- thieno[3 ,4-d]imidazol- 4-yl)pentanamido) hexanamido)pentan- 1-aminium 2,2,2-trifluoroacetate | 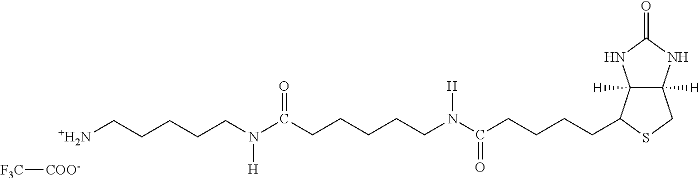 |
| 33 | α-(t-BOC)biocytin 2-(tert- butoxycarbonylamino)- 6-(5-((3aS,6aR)-2- oxohexahydro-1H- thieno[3,4-d]imidazol- 4-yl) pentanamido)hcxanoic acid | 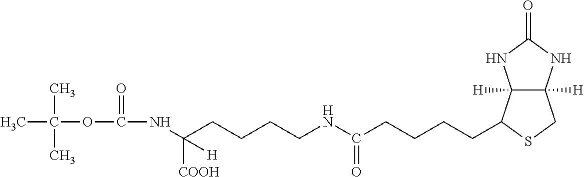 |
| 34 | N-(biotinyl)-N′- (iodoacetyl) ethylenediamine N-(2-(2- iodoacetamido)ethyl)- 5-((3aS,6aR)-2 oxohexahydro-1H- thieno[3,4-d]imidazol- 4-yl) pentanamide | 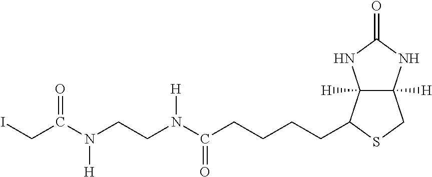 |
| 35 | DNP-X-biocytin-X-SE 2,5-dioxopyrrolidin-1- yl 2-(6-(6-(2,4- dinitrophenylamino) hexanamido) hexanamido)- 6-(6-(5-((3aS,6aR)-2- oxohexahydro-1H- thieno[3,4-d]imidazol- 4-yl) pentanamido) hexanamido) hexanoate | 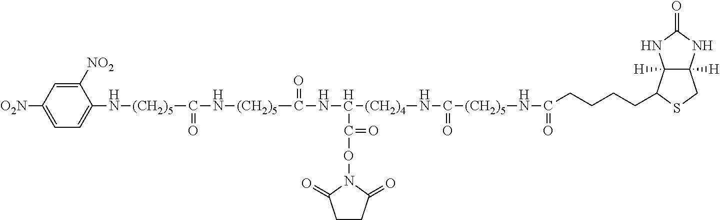 |
| 36 | biotin-X-hydrazide N-(6-hydrazinyl-6- oxohexyl)-5-((3aS,6aR)- 2-oxohexahydro-1H- thieno[3,4-d]imidazol- 4-yl) pentanamide | 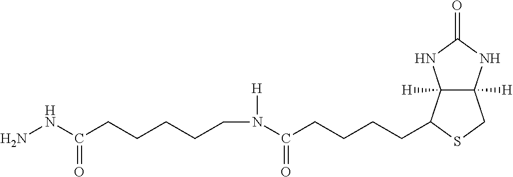 |
| 37 | norbiotinamine hydrochloride 4-((3aS,6aR)-2- oxohexahydro-1H- thieno[3,4-d]imidazol- 4-yl) butan-1-aminium chloride | 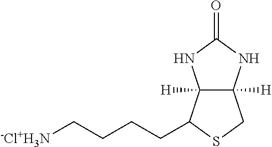 |
| 38 | 3-(N- maleimidylpropionyl) biocytin 2-(3-(2,5-dioxo-2,5- dihydro-1H-pyrrol-1- yl)propanamido)- 6-(5-((3aS,6aR)-2- oxohexahydro-1H- thieno[3,4-d]imidazol- 4-yl) pentanamido)hexanoic acid | 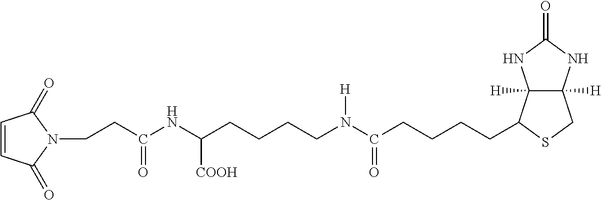 |
| 39 | ARP; N′-(2- (aminooxy)acetyl)- 5-((3aS,6aR)-2- oxohexahydro-1H- thieno[3,4-d]imidazol- 4-yl) pentanehydrazide | 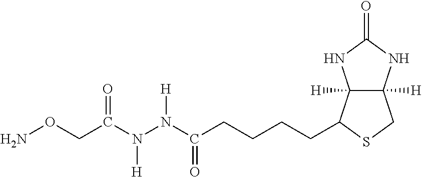 |
| 40 | biotin-1-sulfoxide 5-((3aS,6aR)-2- oxohexahydro-1H- thieno[3,4-d]imidazol- 4-yl) pentanoic acid sulfoxide | 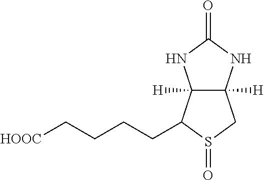 |
| 41 | biotin methyl ester methyl 5-((3aS,6aR)-2- oxohexahydro-1H- thieno[3,4-d]imidazol- 4-yl)pentanoate | 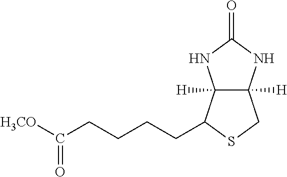 |
| 42 | biotin-maleimide 6-(2,5-dioxo-2,5- dihydro-1H-pyrrol-1- yl)-N′-(5-((3aS,6aR)-2- oxohexahydro-1H- thieno[3,4-d]imidazol- 4-yl)pentanoyl) hexanehydrazide | 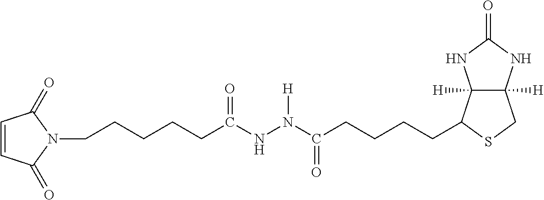 |
| 43 | Biotin- poly(ethyleneglycol) amine aminomethyl polyethylene 5-((3aS,6aR)-2- oxohexahydro-1H- thieno[3,4-d]imidazol- 4-yl) pentanoate | 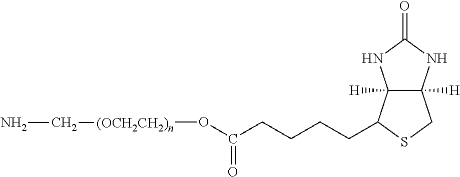 |
| 44 | (+) biotin 4- amidobenzoic acid sodium salt sodium 4-(5-((3aS,6aR)- 2-oxohexahydro-1H- thieno[3,4-d]imidazol- 4-yl) pentanamido) benzoate | 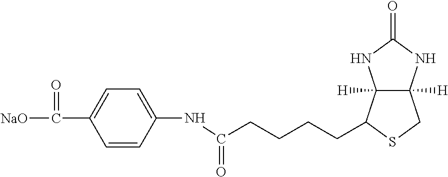 |
| 45 | Biotin 2-N-acetylamino- 2-deoxy-β-D- glucopyranoside ((2R,5S)-3-acetamido- 4,5-dihydroxy-6- (hydroxymethyl)- 2,3,4,5,6- pentamethyltetrahydro- 2H-pyran-2-yl)methyl 5-((3aS,6aR)-2- oxohexahydro-1H- thieno[3,4-d]imidazol- 4-yl) pentanoate | 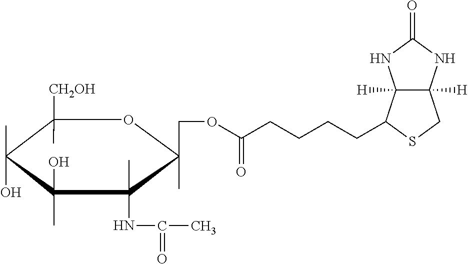 |
| 46 | Biotin-α-D-N- acetylneuraminide (2S,5R)-5-acetamido- 4-hydroxy-3,3,4,5,6- pentamethyl-2-((5- ((3aS,6aR)-2- oxohexahydro-1H- thieno[3,4-d]imidazol- 4-yl) pentanoyloxy)methyl)- 6-(1,2,3- trihydroxypropyl) tetrahydro-2H-pyran- 2-carboxylic acid | 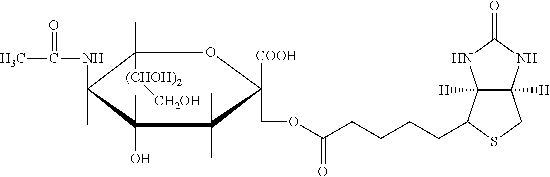 |
| 47 | Biotin-α-L-fucoside ((2R,5S)-3,4,5- trihydroxy-2,3,4,5,6,6- hexamethyltetrahydro- 2H-pyran-2-yl)methyl 5-((3aS,6aR)-2- oxohexahydro-1H- thieno[3,4-d]imidazol- 4-yl)pentanoate | 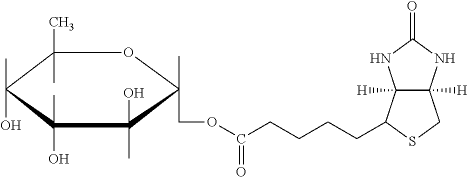 |
| 48 | Biotin lacto-N-bioside See end of table for name | 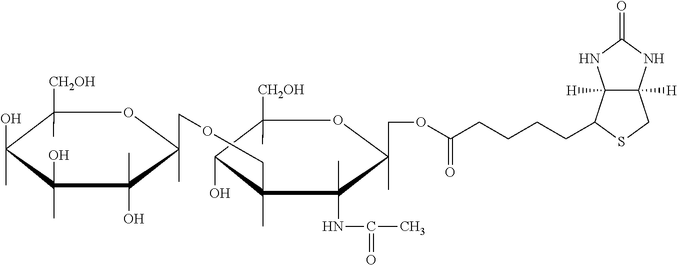 |
| 49 | Biotin-Lewis-A trisaccharide See end of table for name | 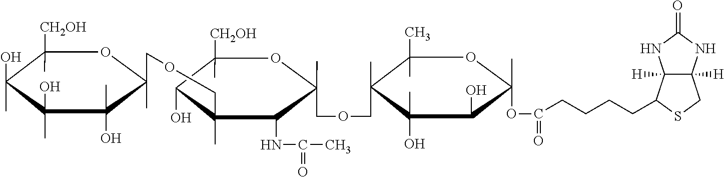 |
| 50 | Biotin-Lewis-Y tetrasaccharide See end of table for name | 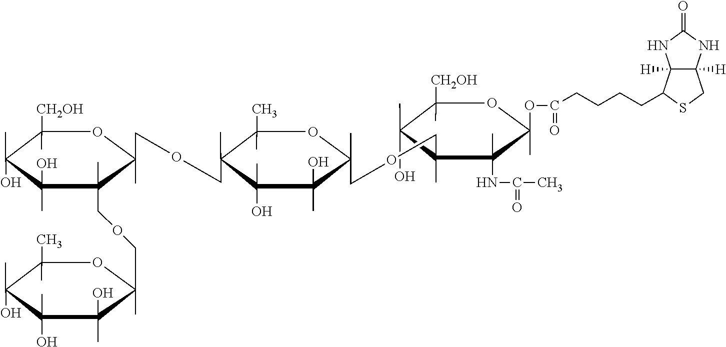 |
| 51 | Biotin-α-D- mannopyranoside ((1R,4R)-2,3,4- trihydroxy-5- (hydroxymethyl)- 1,2,3,4,5- pentamethylcyclohexyl) methyl 5-((3aS,6aR)-2- oxohexahydro-1H- thieno[3,4-d]inaidazol- 4-yl) pentanoate | 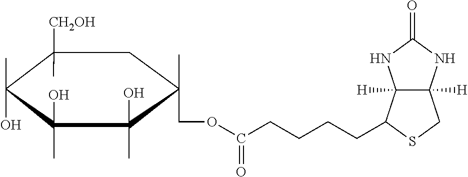 |
| 52 | biotin 6-O-phospho-α- D-mannopyranoside ((2R,5S)-3,4,5- trihydroxy-2,3,4,5,6- pcntamethyl-6- (phosphonooxymethyl) tetrahydro-2H-pyran-2- yl)methyl 5-((3aS,6aR)- 2-oxohexahydro-1H- thieno[3,4-d]imidazol- 4-yl) pentanoate | 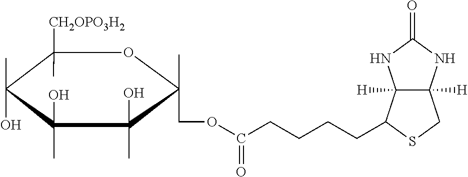 |

Names of Compounds 48-50:
48. ((2R,5S)-3-acetamido-5-hydroxy-6-(hydroxymethyl)-2,3,4,6-tetramethyl-4-(4((((2S,5R)-3,4,5-trihydroxy-6-(hydroxymethyl)-2,3,4,5,6-pentamethyltetrahydro-2H-pyran-2-yl)methoxy)methyl)tetrahydro-2H-pyran-2-yl)methyl 5-((3aS,6aR)-2-oxohexahydro-1H-thieno[3,4-d]imidazol-4-yl)pentanoate ((2R,5S)-3-acetamido-5-hydroxy-6-(hydroxymethyl)-2,3,4,6-tetramethyl-4-((((2S,5R)-3,4,5-trihydroxy-6-(hydroxymethyl)-2,3,4,5,6-pentamethyltetrahydro-2H-pyran-2-yl)methoxy)methyl)tetrahydro-2H-pyran-2-yl)methyl 5-((3aS,6aR)-2-oxohexahydro-1H-thieno[3,4-d]imidazol-4-yl)pentanoate
49. (2R,3R,5S)-5-((((2S,3S,5S)-3-acetamido-5-hydroxy-6-(hydroxymethyl)-2,4,6-trimethyl-4-((((2S,5R)-3,4,5-trihydroxy-6-(hydroxymethyl)-2,3,4,5,6-pentamethyltetrahydro-2H-pyran-2-yl)methoxy) methyl)tetrahydro-2H-pyran-2-yl)methoxy)methyl)-3,4-dihydroxy-2,4,5,6,6-pentamethyltetrahydro-2H-pyran-2-yl 5-((3aS,6aR)-2-oxohexahydro-1H-thieno[3,4-d]imidazol-4-yl)pentanoate
50. (2S,5S)-3-acetamido-4-((((2R,5S)-5-((((2R,5S)-4,5-dihydroxy-6-(hydroxymethyl)-2,3,4,5,6-pentamethyl-3-((((2S,5S)-3,4,5-trihydroxy-2,3,4,5,6,6-hexamethyltetrahydro-2H-pyran-2-yl)methoxy)methyl)tetrahydro-2H-pyran-2-yl)methoxy)methyl)-3,4-dihydroxy-2,3,4,5,6,6-hexamethyltetrahydro-2H-pyran-2-yl)methoxy)methyl)-5-hydroxy-6-(hydroxymethyl)-2,3,4,5,6-pentamethyltetrahydro-2H-pyran-2-yl 5-((3aS,6aR)-2-oxohexahydro-1H-thieno[3,4-d]imidazol-4-yl)pentanoate

Structures of iminobiotin compounds are not shown in Table 2. However, the iminobiotin structures are analogs of the biotin structure where the biotin group is replaced by an iminobiotin group. An example is shown below.

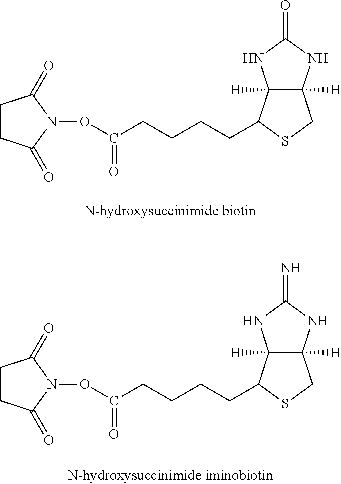

In an embodiment of the invention, metal derived targeting agents may be polymeric or monomeric. Polymeric metal derived targeting agents are fully described in U.S. Pat. No. 7,169,410. Monomeric metal derived targeting agents are described in U.S. Pat. No. 4,603,044. Whether polymeric or monomeric, the compounds generally comprise a metal (typically purchased as an inorganic salt) that may be selected from the transition and inner transition metals or neighbors of the transition metals. The transition and inner transition metals from which the metal is selected include: Sc (scandium), Y (yttrium), La (lanthanum), Ac (actinium), the actinide series; Ti (titanium), Zr (zirconium), Hf (hafnium), V (vanadium), Nb (niobium), Ta (tantalum), Cr (chromium), Mo (molybdenum), W (tungsten), Mn (manganese), Tc (technetium), Re (rhenium), Fe (iron), Co (cobalt), Ni (nickel), Ru (ruthenium), Rh (rhodium), Pd (palladium), Os (osmium), Ir (iridium), and Pt (platinum). The neighbors of the transition metals from which the metal may be selected are: Cu (copper), Ag (silver), Au (gold), Zn (zinc), Cd (cadmium), Hg (mercury), Al (aluminum), Ga (gallium), In (indium), Tl (thallium), Ge (germanium), Sn (tin), Pb (lead), Sb (antimony) and Bi (bismuth), and Po (polonium). Preferably, the metal is chromium.

Non-limiting examples of useful salts include chromium chloride (III) hexahydrate; chromium (III) fluoride tetrahydrate; chromium (III) bromide hexahydrate; zirconium (IV) citrate ammonium complex; zirconium (IV) chloride; zirconium (IV) fluoride hydrate; zirconium (IV) iodide; molybdenum (III) bromide; molybdenum (III) chloride; molybdenum (IV) sulfide; iron (III) hydrate; iron (III) phosphate tetrahydrate, iron (III) sulfate pentahydrate, and the like.

In addition to a metal, the metal derived targeting agent comprises one or more complexing agents. A complexing agent is a compound capable of forming a water insoluble coordination complex with the preferred metal. There are several families of suitable complexing agents.

A complexing agent may be selected from the family of iminodiacetic acids of formula (1) wherein R₁ is loweralkyl, aryl, arylloweralkyl, or a heterocyclic substituent.

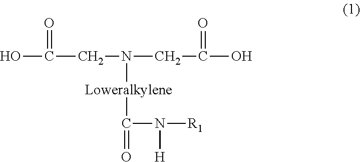

Suitable compounds of formula (1) include:
N-(2,6-diisopropylphenylcarbamoylmethyl)iminodiacetic acid;
N-(2,6-diethylphenylcarbamoylmethyl)iminodiacetic acid;
N-(2,6-dimethylphenylcarbamoylmethyl)iminodiacetic acid;
N-(4-isopropylphenylcarbamoylmethyl)iminodiacetic acid;
N-(4-butylphenylcarbamoylmethyl)iminodiacetic acid;
N-(2,3-dimethylphenylcarbamoylmethyl)iminodiacetic acid;
N-(2,4-dimethylphenylcarbamoylmethyl)iminodiacetic acid;
N-(2,5-dimethylphenylcarbamoylmethyl)iminodiacetic acid;
N-(3,4-dimethylphenylcarbamoylmethyl)iminodiacetic acid;
N-(3,5-dimethylphenylcarbamoylmethyl)iminodiacetic acid;
N-(3-butylphenylcarbamoylmethyl)iminodiacetic acid;
N-(2-butylphenylcarbamoylmethyl)iminodiacetic acid;
N-(4-tertiary butylphenylcarbamoylmethyl)iminodiacetic acid;
N-(3-butoxyphenylcarbamoylmethyl)iminodiacetic acid;
N-(2-hexyloxyphenylcarbamoylmethyl)iminodiacetic acid;
N-(4-hexyloxyphenylcarbamoylmethyl)iminodiacetic acid;
Aminopyrrol iminodiacetic acid;
N-(3-promo-2,4,6-trimethylphenylcarbamoylmethyl)iminodiacetic acid;
Benzimidazole methyl iminodiacetic acid;
N-(3-cyano-4,5-dimethyl-2-pyrrylcarbamoylmethyl)iminodiacetic acid;
N-(3-cyano-4-methyl-5-benzyl-2-pyrrylcarbamoylmethyl)iminodiacetic acid; and
N-(3-cyano-4-methyl-2-pyrrylcarbamoylmethyl)iminodiacetic acid and other derivatives of N-(3-cyano-4-methyl-2-pyrrylcarbamoylmethyl)iminodiacetic acid of formula (2),

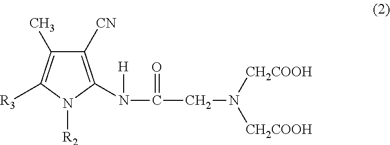

wherein R₂ and R₃ are the following:
R₂ R₃
H iso-C₄H₉
H CH₂CH₂SCH₃
H CH₂C₆H₄-p-OH
CH₃ CH₃
CH₃ iso-C₄H₉
CH₃ CH₂CH₂SCH₃
CH₃ C₆H₅
CH₃ CH₂C₆H₅
CH₃ CH₂C₆H₄-p-OCH₃

Alternatively, the complexing agent may be selected from the family of imino diacid derivatives of formula (3), wherein R₄, R₅, and R₆ are independently selected at each occurrence and may be hydrogen, loweralkyl, aryl, arylloweralkyl, alkoxyloweralkyl, and heterocyclic.

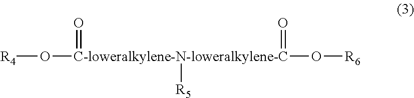

Suitable compounds of formula (3) include: N′-(2-acetylnaphthyl)iminodiacetic acid (NAIDA); N′-(2-naphthylmethyl)iminodiacetic acid (NMIDA); iminodicarboxymethyl-2-naphthylketone phthalein complexone; 3 (3:7a:12a:trihydroxy-24-norchol acyl-23-iminodiacetic acid; benzimidazole methyl iminodiacetic acid; and N-(5,pregnene-3-p-ol-2-oyl carbamoylmethyl)iminodiacetic acid.

The complexing agent may also be selected from the family of amino acids of formula (4),

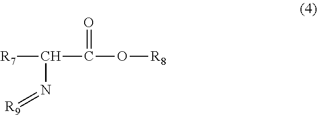

where R₇ is an amino acid side chain; wherein R₈ may be loweralkyl, aryl, and arylloweralkyl; and wherein R₉ is pyridoxylidene.

Suitable amino acids of the formula (4) are aliphatic amino acids, including, but not limited to: glycine, alanine, valine, leucine, isoleucine; hydroxyamino acids, including serine, and threonine; dicarboxylic amino acids and their amides, including aspartic acid, asparagine, glutamic acid, glutamine; amino acids having basic functions, including lysine, hydroxylysine, histidine, arginine; aromatic amino acids, including phenylalanine, tyrosine, tryptophan, thyroxine; and sulfur-containing amino acids, including cysteine and methionine.

The complexing agent may also be selected from amino acid derivatives including, but not limited to (3-alanine-y-amino) butyric acid, O-diazoacetylserine (azaserine), homoserinc, ornithine, citrulline, penicillamine and members of the pyridoxylidene class of compounds. Pyridoxylidene compounds include, but are not limited to: pyridoxylidene glutamate; pyridoxylidene isoleucine; pyridoxylidene phenylalanine; pyridoxylidene tryptophan; pyridoxylidene-5-methyl tryptophan; pyridoxylidene-5-hydroxytryptamine; and pyridoxylidene-5-butyltryptamine.

The complexing agent may likewise be selected from the family of diamines of formula (6):

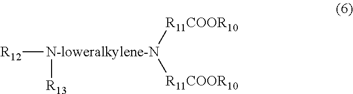

wherein R₁₀ is hydrogen, loweralkyl, or aryl; R¹¹ is loweralkylene or arylloweralky; R₁₂ and R₁₃ are independently selected at each occurrence and may be hydrogen, loweralkyl, alkyl, aryl, arylloweralkyl, acylheterocyclic, toluene, sulfonyl or tosylate.

Examples of suitable diamines of formula (6) include, but are not limited to, ethylenediamine-N,N diacetic acid; ethylenediamine-N,N-bis(-2-hydroxy-5-bromophenyl) acetate; N′-acetylethylenediamine-N,N diacetic acid; N′-benzoyl ethylenediamine-N,N diacetic acid; N′-(p-toluenesulfonyl)ethylenediamine-N,N diacetic acid; N′-(p-t-butylbenzoyl)ethylenediamine-N,N diacetic acid; N′-(benzenesulfonyl)ethylenediamine-N, N diacetic acid; N′-(p-chlorobenzenesulfonyl)ethylenediamine-N,N diacetic acid; N′-(p-ethylbenzenesulfonyl ethylenediamine-N,N diacetic acid; N′-acyl and N′-sulfonyl ethylenediamine-N,N diacetic acid; (p-n-propylbenzenesulfonyl)ethylenediamine-N,N diacetic acid; N′-(naphthalene-2-sulfonyl)ethylenediamine-N,N diacetic acid; and N′-(2,5-dimethylbenzenesulfonyl)ethylenediamine-N,N diacetic acid.

Other, non-limiting examples of complexing compounds or agents include penicillamine; p-mercaptoisobutyric acid; dihydrothioctic acid; 6-mercaptopurine; kethoxal-bis(thiosemicarbazone); Hepatobiliary Amine Complexes, 1-hydrazinophthalazine (hydralazine); sulfonyl-urea; Hepatobiliary Amino Acid Schiff Base Complexes; pyridoxylidene glutamate; pyridoxylidene isoleucine; pyridoxylidene phenylalanine; pyridoxylidene tryptophan; pyridoxylidene 5-methyl tryptophan; pyridoxylidene-5-hydroxytryptamine; pyridoxylidene-5-butyltryptamine; tetracycline; 7-carboxy-p-hydroxyquinoline; phenolphthalein; eosin I bluish; eosin I yellowish; verograffin; 3-hydroxyl-4-formyl-pyridene glutamic acid; Azo substituted iminodiacetic acid; hepatobiliary dye complexes, such as rose bengal; congo red; bromosulfophthalein; bromophenol blue; toluidine blue; and indocyanine green; hepatobiliary contrast agents, such as iodipamide; and ioglycamic acid; bile salts, such as bilirubin; cholgycyliodohistamine; and thyroxine; hepatobiliary thio complexes, such as penicillamine; p-mercaptoisobutyric acid; dihydrothiocytic acid; 6-mercaptopurine; and kethoxal-bis(thiosemicarbazone); hepatobiliary amine complexes, such as 1-hydrazinophthalazine (hydralazine); and sulfonyl urea; hepatobiliary amino acid Schiff Base complexes, including pyridoxylidene-5-hydroxytryptamine; and pyridoxylidene-5-butyltryptamine; hepatobiliary protein complexes, such as protamine; ferritin; and asialo-orosomucoid; and asialo complexes, such as lactosaminated albumin; immunoglobulins G, IgG; and hemoglobin.

Methods of Treating Diabetes, Diabetes Related Ailments, and/or Diseases or Conditions Other than Diabetes or Diabetes Related Ailments Using HDV Insulin

According to one method of the invention, HDV insulin, in an appropriate pharmaceutical formulation, may be administered alone to treat diabetes related ailments and/or diseases other than diabetes or diabetes related ailments. Examples of these ailments and diseases include, but are not limited to, obesity, fatty liver, cardiovascular disease, diabetic coma, diabetic nephrophathy, diabetic neuropathy, erectile dysfunction, metabolic syndrome, diabetic retinopathy, peripheral insulin level elevation, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. HDV insulin may also be administered alone to treat cancer, reduce peripheral insulin levels, affect weight loss, or to assist with weight management. Similarly, HDV insulin may be administered before, during, or after surgery as an anti-stress metabolic enhancement agent.

According to another method of the invention, HDV insulin in an appropriate pharmaceutical formulation, may be co-administered, i.e. given as a combination therapy, with one or more additional therapeutic agents to treat diabetes, diabetes related ailments, and/or diseases or conditions other than diabetes or diabetes related ailments.

Examples of these ailments and diseases include, but are not limited to, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. HDV insulin may also be co-administered with one or more additional therapeutic agents to treat cancer, reduce peripheral insulin levels, affect weight loss, or to assist with weight management, Similarly, HDV insulin may be co-administered with one or more additional therapeutic agents before, during, or after surgery as an anti-stress metabolic enhancement agent.

Examples of appropriate additional therapeutic agents appropriate for co-administration include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, serotonin-norepinephrine reuptake inhibitors, and cannabinoid (“CBI”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

Although a physician will be able to select the appropriate dose for a given patient, the range of insulin that may be delivered by HDV insulin in any of the above described methods is from about 1 to about 40 units, but may be any number of units including 2, 3, 4, 5, 10, 15, 20, 25, 30, 35, or other whole or partial increment therebetween. A given formulation may also exceed 40 units.

The above described methods may be undertaken at any time of the day or night as well as any time pre- or post-prandially, or during the course of a meal. Generally, HDV insulin is not given orally. When HDV insulin is co-administered with one or more additional therapeutic agents, the one or more additional therapeutic agents may be administered according to any acceptable route of administration appropriate for the given therapeutic agent.

### HDV Insulin Pharmaceutical Composition

The present invention further includes an HDV insulin pharmaceutical composition comprising HDV insulin and one or more therapeutic agents not associated with the HDV insulin. Examples of therapeutic agents include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, Serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

This pharmaceutical composition may be prepared by first preparing HDV insulin according to the general procedure set forth herein and then formulating the HDV insulin with one or more therapeutic agents. The one or more therapeutic agents are not associated with the HDV insulin. The HDV insulin pharmaceutical composition may then be administered to a patient in need thereof.

Methods of Treating Diabetes, Diabetes Related Ailments, and/or Diseases or Conditions Other than Diabetes or Diabetes Related Ailments Using a Pharmaceutical Composition Comprising HDV Insulin and One or More Therapeutic Agents not Associated with HDV Insulin

According to a method of the invention, a pharmaceutical composition comprising HDV insulin and one or more additional therapeutic agents not associated with HDV insulin may be administered to a patient in need thereof to treat diabetes, diabetes related ailments, and/or diseases other than diabetes or diabetes related ailments.

Examples of these ailments and diseases include, but are not limited to, diabetes, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. A pharmaceutical composition comprising HDV insulin and one or more additional therapeutic agents not associated with HDV insulin may also be administered to a patient in need thereof to treat cancer, reduce peripheral insulin levels, affect weight loss, or to assist with weight management. Similarly, HDV insulin and one or more additional therapeutic agents not associated with HDV insulin may be administered before, during, or after surgery as an anti-stress metabolic enhancement agent.

Examples of appropriate additional therapeutic agents include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination stain therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

Although a physician will be able to select the appropriate dose for a given patient, the range of insulin that may be delivered by HDV insulin in any of the above described methods is from about 1 to about 40 units, but may be any number of units including 2, 3, 4, 5, 10, 15, 20, 25, 30, 35, or other whole or partial increment therebetween. A given formulation may also exceed 40 units.

The above described methods may be undertaken at any time of the day or night as well as any time pre- or post-prandially, or during the course of a meal. Generally, a pharmaceutical composition comprising HDV insulin and one or more additional therapeutic agents not associated with HDV insulin is not given orally. Additional limitations on the administrability of the pharmaceutical composition of the invention comprising HDV insulin and one or more additional therapeutic agents not associated with HDV insulin may be imposed by any limitations on administration of the one or more therapeutic agents comprising the pharmaceutical composition.

Pharmaceutical Formulations of HDV Insulin and a Pharmaceutical Composition Comprising HDV Insulin and One or More Therapeutic Agents not Associated with HDV Insulin

Pharmaceutical formulations of HDV insulin and the pharmaceutical composition comprising HDV insulin and one or more therapeutic agents not associated with HDV insulin may be prepared by any method known or hereafter developed in the art of pharmacology. In general, such preparatory methods include the step of bringing HDV insulin or HDV insulin and one or more therapeutic agents not associated with HDV insulin into contact with a carrier or one or more other accessory ingredients, and then, if necessary or desirable, shaping or packaging the product into a desired single- or multi-dose unit.

Although a pharmaceutical formulation of HDV insulin and a pharmaceutical composition comprising HDV insulin and one or more therapeutic agents not associated with HDV insulin are principally suitable for ethical administration to humans, it will be understood by the skilled artisan that these formulations are generally suitable for administration to animals of all sorts. Modification of the pharmaceutical compositions of the invention suitable for administration to humans in order to render the agents suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical formulation of HDV insulin may be prepared, packaged, or sold in bulk, as a single unit dose, or as a plurality of single unit doses. As used herein, a “unit dose” is discrete amount of HDV insulin. The amount of HDV insulin may be a whole dosage or a convenient fraction of such a dosage such as, for example, one-half or one-third of such a dosage.

A pharmaceutical composition comprising HDV insulin and one or more therapeutic agents not associated with HDV insulin may be prepared, packaged, or sold in bulk, as a single unit dose, or as a plurality of single unit doses. As used herein, a “unit dose” is discrete amount of the pharmaceutical composition comprising a predetermined amount of HDV insulin and one or more additional therapeutic agents. The amount of HDV insulin and one or more additional therapeutic agents is generally equal to the dosage of HDV insulin and one or more additional therapeutic agents which would typically be administered to a subject if given separately or a convenient fraction of such a dosage such as, for example, one-half or one-third of such a dosage.

In a pharmaceutical formulation of HDV insulin, the relative amount of the HDV insulin and the pharmaceutically acceptable carrier will vary, depending upon the identity, size, and condition of the subject treated and further depend upon the route by which the pharmaceutical formulation is to be administered. By way of example, the composition may comprise between 0.1% and 99.9% (w/w) of HDV insulin.

In a formulation of the pharmaceutical composition comprising HDV insulin and one or more therapeutic agents not associated with HDV insulin, the relative amounts of HDV insulin and one or more additional therapeutic agents, the pharmaceutically acceptable carrier, and any additional ingredients will vary, depending upon the identity, size, and condition of the subject treated and further depending upon the route by which the composition is to be administered. By way of example, the composition may comprise between 0.1% and 99.9% (w/w) of HDV insulin and between 99.9% and 0.1% of one or more additional therapeutic agents not associated with HDV insulin.

A pharmaceutical composition comprising HDV insulin and one or more therapeutic agents not associated with HDV insulin may further comprise one or more additional pharmaceutically active ingredients. Particularly contemplated additional active ingredients include anti-emetics and scavengers such as cyanide and cyanate scavengers.

Similarly, a pharmaceutical formulation of HDV insulin may be formulated with one or more additional pharmaceutically active ingredients. Particularly contemplated additional active ingredients include anti-emetics and scavengers such as cyanide and cyanate scavengers.

Controlled- or sustained-release formulations of HDV insulin or of the pharmaceutical composition comprising HDV insulin and one or more therapeutic agents not associated with HDV insulin may be made using conventional technology.

Liquid formulations of HDV insulin or a pharmaceutical composition comprising HDV insulin and one or more therapeutic agents not associated with HDV insulin may be prepared, packaged, and sold either in liquid form or in the form of a dry product intended for reconstitution with water or another suitable vehicle prior to use.

Liquid suspensions may be prepared using conventional methods to achieve suspension of the constituents in an aqueous vehicle. Aqueous vehicles include, for example, water and isotonic saline. Oily vehicles may only be used to the extent that such solvents are not incompatible with HDV insulin and do not disturb the assembled structure of the lipid components. To the extent that an oily suspension is not incompatible with HDV insulin, an oily suspension may further comprise a thickening agent.

Liquid suspensions may further comprise one or more additional ingredients to the extent that said ingredients do not disrupt the HDV structures. Examples of additional ingredients include, but are not limited to, suspending agents, dispersing or wetting agents, emulsifying agents, demulcents, preservatives, buffers, salts, flavorings, coloring agents, and sweetening agents.

Known suspending agents include, but are not limited to, sorbitol syrup, sodium alginate, polyvinylpyrrolidone, gum tragacanth, gum acacia, and cellulose derivatives such as sodium carboxymethylcellulose, methylcellulose, hydroxypropylmethylcellulose.

Known emulsifying agents include, but are not limited to acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin.

Powdered and granular formulations of HDV insulin or a pharmaceutical composition comprising HDV insulin and one or more therapeutic agents not associated with HDV insulin may be prepared using known methods. Such formulations may be administered directly to a subject, used, for example to prepare an aqueous suspension by addition of an aqueous vehicle thereto. This formulation may further comprise one or more of dispersing or wetting agent, a suspending agent, and a preservative. Additional excipients, such as fillers or coloring agents, may also be included in these formulations.

As used herein, “parenteral administration” of a pharmaceutical formulation of HDV insulin or a pharmaceutical composition comprising HDV insulin and one or more therapeutic agents not associated with HDV insulin includes any route of administration characterized by physical breaching of a tissue of a subject and administration of the pharmaceutical composition through the breach in the tissue. Parenteral administration thus includes, but is not limited to, injection of a pharmaceutical formulation of HDV insulin or a pharmaceutical composition comprising HDV insulin and one or more therapeutic agents not associated with HDV insulin, by application of a pharmaceutical formulation of HDV insulin or a pharmaceutical composition comprising HDV insulin and one or more therapeutic agents not associated with HDV insulin through a surgical incision, by application of a pharmaceutical formulation of HDV insulin or a pharmaceutical composition comprising HDV insulin and one or more therapeutic agents not associated with HDV insulin through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of HDV insulin or a pharmaceutical composition comprising HDV insulin and one or more therapeutic agents not associated with HDV insulin for parenteral administration comprise HDV insulin and, as appropriate, one or more additional therapeutic agents not associated with HDV insulin, combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi dose containers containing a preservative. Formulations for parenteral administration include, but are not limited to, suspensions, emulsions in oily or aqueous vehicles (subject to the stability of HDV insulin in said oily vehicle), pastes, and implantable sustained-release or biodegradable formulations. Such formulations may further comprise one or more additional ingredients including, but not limited to, suspending, stabilizing, or dispersing agents. In one embodiment of a formulation for parenteral administration, HDV insulin or a pharmaceutical composition comprising HDV insulin and one or more therapeutic agents not associated with HDV insulin are provided in solid (i.e. powder or granular) form for reconstitution with a suitable vehicle (e.g. sterile pyrogen free water) prior to parenteral administration of the reconstituted pharmaceutical.

A pharmaceutical formulation of HDV insulin or pharmaceutical composition comprising HDV insulin and one or more therapeutic agents not associated with HDV insulin may be prepared, packaged, or sold in the form of a sterile injectable aqueous suspension. This suspension may be formulated according to the known art, and may comprise, in addition to HDV insulin or a pharmaceutical composition comprising HDV insulin and one or more therapeutic agents not associated with HDV insulin, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, and isotonic sodium chloride solution. Other parentally-administrable formulations which are useful include those which comprise HDV insulin or a pharmaceutical composition comprising HDV insulin and one or more therapeutic agents not associated with HDV insulin in microcrystalline form for ultrasound-released delivery or as a component of a biodegradable polymer system. A pharmaceutical formulation of HDV insulin or a pharmaceutical composition comprising HDV insulin and one or more therapeutic agents not associated with HDV insulin for sustained release or implantation may comprise pharmaceutically acceptable polymeric or hydrophobic materials such as an emulsion, an ion exchange resin, a sparingly soluble polymer, or a sparingly soluble salt.

### Kits Including HDV Insulin

The present invention also includes a kit containing a pharmaceutical formulation of HDV insulin or a pharmaceutical composition comprising HDV insulin and one or more therapeutic agents not associated with HDV insulin. Alternatively, a kit may contain a pharmaceutical formulation of HDV insulin and one or more therapeutic agents for co-administration with a pharmaceutical formulation of HDV insulin.

The above described kits further include instructional material which describes administering the contents of the kit to a mammal. As used herein, an “instructional material” includes a publication, a recording, a diagram, or any other medium of expression which can be used to communicate the usefulness of the composition of the invention in the kit for effecting alleviation of the various diseases or disorders recited herein.

Optionally, or alternatively, the instructional material may describe one or more methods of alleviating the diseases or disorders in a cell or a tissue of a mammal. The instructional material of the kit may, for example, be affixed to a container which contains the invention or be shipped together with a container which contains the invention, Alternatively, the instructional material may be shipped separately from the container with the intention that the instructional material and the compound be used cooperatively by the recipient.

A kit may include a pharmaceutical formulation of HDV insulin or a pharmaceutical composition comprising HDV insulin and one or more therapeutic agents not associated with HDV insulin packaged in a container for convenient QD, BID, TID, qXh (where X is an integer from 1 to 24), weekly, or monthly administration. A kit may further include one or more packages containing sufficient amounts of a pharmaceutical formulation of HDV insulin or a pharmaceutical composition comprising HDV insulin and one or more therapeutic agents not associated with HDV insulin for administration for 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any increment therebetween.

A kit containing a pharmaceutical formulation of HDV insulin as described above may further include one or more therapeutic agents for co-administration with the pharmaceutical formulation of HDV insulin, packaged in one or more packages either with or separate from the pharmaceutical formulation of HDV insulin, in the manner noted above, i.e. for convenient QD, BID, TID, qXh (where X is an integer from 1 to 24), weekly, or monthly administration or with enough one or more therapeutic agent for administration for 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any and all increments therebetween.

### Oral HDV

Oral HDV insulin is an orally bioavailable form of HDV insulin that functions by chaperoning insulin through the lumen of the gut into the portal blood flow and finally on to the systemic circulation. Without wishing to be bound to one particular theory, it is believed that oral HDV insulin inserts into intercellular gaps and passes through the mammalian gut into the portal circulation. In certain embodiments, oral HDV insulin may be targeted to specific cellular or extra-cellular receptors via one or more targeting agents. Thus, insulin administered as oral HDV insulin, is delivered to a particular cell or class of cells, such as, for example, hepatocytes.

In a typical embodiment, oral HDV insulin comprises at least one insulin, gelatin, and additional constituents. The additional constituents comprise a dynamically sized liposome, liposome fragment, and lipid particle, wherein the lipid particle comprises at least one lipid component and the liposome or liposome fragment comprise at least two lipid components. Oral HDV insulin further comprises at least one insulin and, optionally, at least one targeting agent. The gelatin actively reversibly interacts with insulin and/or one or more of the constituents of oral HDV insulin.

Traditionally, liposome, liposome fragments, and lipid particles comprised of amphipathic materials have been limited to a lower size distribution of about 40 nanometers. This limit was believed to be a function of the collective sizes of the constituent lipids (phospholipids, cholesterols, diatkylphosphates, etc.) that constituted the membrane structure.

The constituents of oral HDV insulin, however, demonstrate heretofore unobserved dynamic sizing and size elasticity. Specifically, constituents of oral HDV insulin, exist in a dynamic equilibrium in aqueous media wherein the constituents, on average, fluctuate in size from about 6 nanometers to about 60 nanometers in diameter. At any given time, anywhere from about 5% to about 50% of the constituents exhibit an average diameter of about 20 nanometers or less. Due to the nearly constant fluctuations in sizes, the constituents of oral HDV insulin cannot be physically separated by traditional fractionating means to form discrete populations of differently sized structures. The constituents of oral HDV insulin may be, but are not limited to, a liposome, a liposome fragment, and a lipid particle.

Without wishing to be bound by any particular theory, it is believed that constituents having diameters of 20 nanometers or less are sufficiently small to pass through intercellular gaps, thus enabling transport of associated insulin from the lumen of the gut into the portal blood.

The associated insulin is preferably noncovalently bound to one or more constituents of oral HDV insulin. Insulin may, however, be bound covalently. In embodiments wherein the associated insulin is bound covalently, insulin may be bound to a chemical group that can be functionalized. Examples of functionalizable groups include, but are not limited to, hydroxy, amino, carboxy, and amido groups.

Alternatively, and more preferably, oral HDV insulin binds to insulin via non-covalent interactions.

A constituent of oral HDV insulin comprises one or more lipid components and an optional targeting agent. An embodiment comprising a single unit or multiple units of a single lipid component is referred to herein as a “lipid particle.” An embodiment comprising two or more different lipid components and an optional targeting agent is classified as a liposome or liposome fragment, depending upon the nature of the resulting structure. Lipid components are selected from the group lipids used to prepare oral HDV insulin, examples of which are provided in Table 1.

By way of non-limiting examples, the constituents of oral HDV insulin may be formed from lipid components mixed in accordance with the following: approximately 61 mole percent 1,2 distearoyl-sn-glycero-3-phosphocholine, approximately 22 mole percent dihexadecyl phosphate, and approximately 16 mole percent cholesterol. In embodiments wherein a constituent incorporates a targeting agent, the above noted mixture may further include from about 1 to about 2 mole percent of at least one targeting agent, with the amounts of other lipid components reduced to maintain the molar ratio of components set forth above.

In another embodiment, any of the constituents of oral HDV may also include at least one diagnostic agent in combination with or in place of a targeting agent. Examples of diagnostic agents include diagnostic contrast agents such as, but not limited to, gold and a gadolinium. Other diagnostic agents include radioactive materials such as radioactive isotopes of common atoms including, but not limited to, ¹³C, ⁶⁸Ge, ¹⁸F, and ¹²⁵I. These contrast and radioactive agents are preferably covalently attached to a lipid component and/or targeting agent using known techniques in synthetic organic chemistry. Alternatively, and where chemically appropriate, the diagnostic agent may be bound to a ligand such as DADO (2′-deoxyadenosine), which is itself covalently attached to a lipid component or targeting agent using known techniques in synthetic organic chemistry.

Stability of Insulin Associated with Oral HDV Insulin

Although constituent members of oral HDV insulin are formulated in aqueous media, the constituent members of oral HDV insulin do not exhibit long term stability in water. Specifically, water aids hydrolysis of any acyl chains present in any of the lipid components of the compositional constituents. The aqueous environment also allows for the ready oxidation of any unsaturated acyl chains present in any of the lipid components. In a preferred embodiment of the present invention, the constituents of oral HDV insulin, and insulin, are protected for long term storage via interaction with a proteoglycan such as a modified collagen, known generically as dry granulated gelatin. Dry granulated gelatin, when contacted with an aqueous suspension of oral HDV insulin, reacts with water, and stabilizes the constituent/insulin construct.

The reaction of dried granulated gelatin with an aqueous suspension of oral HDV insulin, results in a semi-solid colloidal gel that shields the insulin and associated constituents from direct interaction with water. Any water not associated with gelatin is slowly evaporated via refrigerated storage at about 2° to about 8° C. The water may, however, be removed via techniques including, but not limited, freeze drying and spray drying.

This results in a pellet like “dry” insulin/constituent/gelatin complex. In this complex, the constituent elements are partially dehydrated in a reversible manner and sequestered by the proteinaceous lattice of dry gelatin. This sequestration is enabled by structured water, structured lipid and structured gelatin all interacting through hydrogen bonding, ionic bonding, van der Waal's interactions, and hydrophobic bonding between the lipid components, water, and protein structures, i.e., insulin. This evidences that gelatin is not acting as an emulsifying or suspending agent. As a result, the “dry” pellet is stable for long term storage because the activity of water has been mitigated. These pellets can be further processed to a granulated or free-flowing powder for final capsule filling or tabletting, while maintaining their stability and efficacy.

Upon oral administration to a patient, the “dry” pellet becomes hydrated and once again assumes a semi-solid colloidal gel state. Upon further exposure to the gastric environment, the gel becomes liquid as gelatin is solubilized. Once the gelatin is completely solubilized, the constituent members of oral HDV insulin rehydrate, resulting in the formation of a new suspension of oral HDV insulin within the gastric environment that may then be absorbed into the portal blood flow.

It is important to realize that the role of gelatin in this aspect of the invention is as an active stabilizer of the composition and not an inert filler as is commonly found in oral formulations of many other pharmaceutical compositions. That said, the additional use of gelatin as an inert filler in addition to the aforementioned use is also contemplated.

Although gelatin is used in a preferred embodiment of the invention, other gelatin like compounds may be used as well. Examples of agents that will act as active stabilizers include, but are not limited to, acacia (gum arabic), agar (agar-agar; vegetable gelatin; gelosa; Chinese or Japanese gelatin), alginic acid, sodium alginate (alginic acid; sodium salt; algin; Manucol; Norginc; Kelgin), carbomer (carboxypolymethylene), carrageenan, carboxymethylcellulose sodium (carbose D; carboxymethocel S; CMC; cellulose gum), powdered cellulose (Degussa), hydroxyethyl cellulose (cellulose; 2-hydroxyethyl ether; Cellosize; Natrosol), hydroxypropyl cellulose (cellulose; 2-hydroxypropyl ether; Klucel), hydroxypropyl methylcellulose (cellulose; 2-hydroxypropyl methyl ether), methycellulose (cellulose; methyl ether Methocel), povidone (2-pyrrolidinone; 1-ethenyl-; homopolymer; polyvinylpyrrolidone), tragacanth (gum tragacanth; Hog Gum; Goat's Thorn), and xanthan gum (Keltrol). Like gelatin, and where appropriate, these compounds may also be used as inert fillers.

Methods of Treating Diabetes, Diabetes Related Ailments, and/or Diseases Other than Diabetes or Diabetes Related Ailments Using Oral HDV Insulin

According to one method of the invention, oral HDV insulin, in an appropriate pharmaceutical formulation, may be administered alone to treat diabetes related ailments and/or diseases other than diabetes or diabetes related ailments. Examples of these diseases include, but are not limited to, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. Oral HDV insulin may also be administered alone to treat cancer, reduce peripheral insulin levels, affect weight loss, or to assist with weight management. Additionally, oral HDV insulin may be administered before, during, or after surgery as an anti-stress metabolic enhancement agent.

According to another method of the invention, oral HDV insulin, in an appropriate pharmaceutical formulation, may be co-administered, i.e. given as a combination therapy, with one or more additional therapeutic agents to treat diabetes, diabetes related ailments, and/or diseases other than diabetes or diabetes related ailments.

Examples of these ailments and diseases include, but are not limited to, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. Oral HDV insulin in an appropriate pharmaceutical formulation may also be co-administered with one or more additional therapeutic agents to reduce peripheral insulin levels, affect weight loss, or to assist with weight management. Additionally, oral HDV insulin in an appropriate pharmaceutical formulation may be co-administered with one or more additional therapeutic agents before, during, or after surgery as an anti-stress metabolic enhancement agent

Examples of appropriate additional therapeutic agents for co-administration include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase hOmolog 1 (DGAT-1) inhibitors, dopamine receptor agonists, dual-acting peptide-GLP-1 and glucagons receptor agonists, EGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

Although a physician will be able to select the appropriate dose for a given patient, the range of insulin that may be delivered by oral HDV insulin is from about 1 to about 40 units, but may be any number of units including 2, 3, 4, 5, 10, 15, 20, 25, 30, 35, or other whole or partial increment therebetween. A given formulation may also exceed 40 units.

The above described methods may be undertaken at any time of the day or night as well as any time pre- or postprandially, or during the course of a meal. Generally, oral HDV insulin is given orally. Any co-administered therapeutic agents may be administered via any appropriate route for that therapeutic agent.

### Oral HDV Pharmaceutical Composition

The present invention further includes an oral HDV pharmaceutical composition comprising oral HDV insulin and one or more therapeutic agents not associated with the oral HDV insulin. Examples of therapeutic agents include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, Serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor Mockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropie receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

This pharmaceutical composition may be prepared by first preparing oral HDV insulin according to the general procedure set forth herein and then formulating the oral HDV insulin with one or more therapeutic agents. The one or more therapeutic agents are not associated with the oral HDV insulin. The oral HDV insulin pharmaceutical composition may then be administered to a patient in need thereof.

Methods of Treating Diabetes, Diabetes Related Ailments, and/or Diseases Other than Diabetes or Diabetes Related Ailments Using an Oral HDV Pharmaceutical Composition

According to another method of the invention, a pharmaceutical composition comprising oral HDV insulin and one or more additional therapeutic agents not associated with oral HDV insulin may be administered to a patient in need thereof to treat diabetes, diabetes related ailments, and/or diseases other than diabetes or diabetes related ailments.

Examples of these ailments and diseases include, but are not limited to, diabetes, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemiareperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity. A pharmaceutical composition comprising oral HDV insulin and one or more additional therapeutic agents not associated with oral HDV insulin may also be administered to treat cancer, reduce peripheral insulin levels, affect weight loss, or to assist with weight management. Additionally, the pharmaceutical composition may be administered before, during, or after surgery as an anti-stress metabolic enhancement agent.

Examples of appropriate additional therapeutic agents not associated with oral HDV insulin include, but are not limited to, α-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARcrly modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

Although a physician will be able to select the appropriate dose for a given patient, the range of insulin that may be delivered by oral HDV insulin is from about 1 to about 40 units, but may be any number of units including 2, 3, 4, 5, 10, 15, 20, 25, 30, 35, or other whole or partial increment therebetween. A given formulation may also exceed 40 units.

The above described methods may be undertaken at any time of the day or night as well as any time pre- or post-prandially, or during the course of a meal. Generally, a pharmaceutical composition comprising oral HDV insulin and one or more additional therapeutic agents not associated with oral HDV insulin is given orally. Additional limitations on the administrability of the pharmaceutical composition comprising oral HDV insulin and one or more additional therapeutic agents not associated with oral HDV insulin may be imposed by any limitations on administration of the one or more therapeutic agents comprising the pharmaceutical composition.

### Formulations of Oral HDV Insulin

A pharmaceutical formulation of oral HDV insulin and a pharmaceutical composition comprising oral HDV insulin and one or more additional therapeutic agents not associated with oral HDV insulin described herein may be prepared by any method known or hereafter developed in the art of pharmacology. In general, such preparatory methods include the step of bringing oral HDV insulin into contact with a carrier or one or more other accessory ingredients, and then, if necessary or desirable, shaping or packaging the product into a desired single- or multi-dose unit.

Alternatively, such preparatory methods include the step of bringing a pharmaceutical composition comprising oral HDV insulin and one or more additional therapeutic agents not associated with oral HDV insulin into contact with a carrier or one or more other accessory ingredients, and then, if necessary or desirable, shaping or packaging the product into a desired single- or multi-dose unit.

Although a pharmaceutical formulation of oral HDV insulin and a pharmaceutical composition comprising oral HDV insulin and one or more additional therapeutic agents not associated with oral HDV insulin are principally suitable for ethical administration to humans, it will be understood by the skilled artisan that both oral HDV insulin and a pharmaceutical composition comprising oral HDV insulin and one or more additional therapeutic agents not associated with oral HDV insulin are generally suitable for administration to animals of all sorts. Modification of a pharmaceutical formulation of oral HDV insulin or a pharmaceutical composition comprising oral HDV insulin and one or more additional therapeutic agents not associated with oral HDV insulin in order to render the agents suitable for administration to various animals is well understood, and the ordinarily skilled veterinary pharmacologist can design and perform such modification with merely ordinary, if any, experimentation.

Subjects to which administration of a pharmaceutical formulation of oral HDV insulin and a pharmaceutical composition comprising oral HDV insulin and one or more additional therapeutic agents not associated with oral HDV insulin is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical formulation of oral HDV insulin or a pharmaceutical composition comprising oral HDV insulin and one or more additional therapeutic agents not associated with oral HDV insulin that are useful in the methods of the invention may be prepared, packaged, or sold in formulations suitable for oral, parenteral, buccal, or another route of administration.

A pharmaceutical formulation of oral HDV insulin may be prepared, packaged, or sold in bulk, as a single unit dose, or as a plurality of single unit doses. As used herein, a “unit dose” is discrete amount of oral HDV insulin. The amount of oral HDV insulin may be a whole dosage or a convenient fraction of such a dosage such as, for example, one-half or one-third of such a dosage.

A pharmaceutical composition comprising oral HDV insulin and one or more therapeutic agents not associated with oral HDV insulin may be prepared, packaged, or sold in bulk, as a single unit dose, or as a plurality of single unit doses. As used herein, a “unit dose” is discrete amount of the pharmaceutical composition comprising a predetermined amount of oral HDV insulin and one or more additional therapeutic agents. The amount of oral HDV insulin and one or more additional therapeutic agents is generally equal to the dosage of oral HDV insulin and one or more additional therapeutic agents which would typically be administered to a subject if given separately or a convenient fraction of such a dosage such as, for example, one-half or one-third of such a dosage.

In a pharmaceutical formulation of oral HDV insulin, the relative amount of the oral HDV insulin and the pharmaceutically acceptable carrier will vary, depending upon the identity, size, and condition of the subject treated and further depend upon the route by which the pharmaceutical formulation is to be administered. By way of example, the composition may comprise between 0.1% and 99.9% (w/w) of oral HDV insulin.

In a pharmaceutical composition comprising oral HDV insulin and one or more therapeutic agents not associated with oral HDV insulin, the relative amounts of oral HDV insulin and one or more additional therapeutic agents, the pharmaceutically acceptable carrier, and any additional ingredients will vary, depending upon the identity, size, and condition of the subject treated and further depending upon the route by which the composition is to be administered. By way of example, the composition may comprise between 0.1% and 99.9% (w/w) of oral HDV insulin and between 99.9% and 0.1% of one or more additional therapeutic agents not associated with oral HDV insulin.

In addition to oral HDV insulin or a pharmaceutical composition comprising oral HDV insulin and one or more therapeutic agents not associated with oral HDV insulin, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of the pharmaceutical composition of the invention may be made using conventional technology.

For a pharmaceutical composition comprising oral HDV insulin or a pharmaceutical composition comprising oral HDV insulin and one or more therapeutic agents not associated with oral HDV insulin, the pharmaceutical composition may be prepared, packaged, or sold in the form of a discrete solid dose unit including, but not limited to, a tablet, a hard or soft capsule, a cachet, a troche, or a lozenge, each containing a predetermined amount of oral HDV insulin, and as appropriate, one or more therapeutic agents not associated with HDV insulin. Other formulations suitable for oral administration include, but are not limited to, a powdered or granular formulation, aqueous suspensions, or emulsions.

A tablet comprising oral HDV insulin or a pharmaceutical composition comprising oral HDV insulin and one or more therapeutic agents not associated with oral HDV insulin, for example, be made by compressing or molding oral HDV insulin or a pharmaceutical composition comprising oral HDV insulin and one or more therapeutic agents not associated with oral HDV insulin optionally with one or more additional ingredients. Compressed tablets may be prepared by compressing, in a suitable device, the composition in a free-flowing form such as a powder or granular preparation, optionally mixed with one or more of a binder, a lubricant, an excipient, a surface active agent, and a dispersing agent. Molded tablets may be made by molding, in a suitable device, oral HDV insulin or a pharmaceutical composition comprising oral HDV insulin and one or more therapeutic agents not associated with oral HDV insulin, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulfate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the pharmaceutical composition or insulin associated with oral HDV insulin. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising a pharmaceutical formulation of oral HDV insulin or a pharmaceutical composition comprising oral HDV insulin and one or more therapeutic agents not associated with oral HDV insulin may be made using a physiologically degradable composition, such as gelatin. Such hard capsules comprise oral HDV insulin or a pharmaceutical composition comprising oral HDV insulin and one or more therapeutic agents not associated with oral HDV insulin, and may further comprise additional ingredients including, for example, an inert solid diluent such as calcium carbonate, calcium phosphate, kaolin or cellulose acetate hydrogen phthalate.

Soft gelatin capsules comprising a pharmaceutical formulation of oral HDV insulin or a pharmaceutical composition comprising oral HDV insulin and one or more therapeutic agents not associated with oral HDV insulin, may be made using a physiologically degradable composition, such as gelatin.

Liquid pharmaceutical formulations of oral HDV insulin or a pharmaceutical composition comprising oral HDV insulin and one or more therapeutic agents not associated with oral HDV insulin, may be packaged and sold either in liquid form or in the form of a dry product intended for reconstitution with water or another suitable vehicle prior to use. Suitable aqueous vehicles include, for example, water and isotonic saline. Oily vehicles may only be used to the extent that such solvents are not incompatible with oral HDV insulin. To the extent that an oily suspension is not incompatible with oral HDV insulin, an oily suspension may further comprise a thickening agent.

Liquid suspensions may further comprise one or more additional ingredients to the extent that said ingredients do not disrupt the structure of oral HDV. Examples of additional ingredients include, but are not limited to, suspending agents, dispersing or wetting agents, emulsifying agents, demulcents, preservatives, buffers, salts, flavorings, coloring agents, and sweetening agents.

Known suspending agents include, but are not limited to, sorbitol syrup, sodium alginate, polyvinylpyrrolidone, gum tragacanth, gum acacia, and cellulose derivatives such as sodium carboxymethylcellulose, methylcellulose, hydroxypropylmethylcellulose.

Known emulsifying agents include, but are not limited to acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin.

Powdered and granular pharmaceutical formulations of oral HDV insulin or a pharmaceutical composition comprising oral HDV insulin and one or more therapeutic agents not associated with oral HDV insulin may be prepared using known methods. Such formulations may be administered directly to a subject, used, for example, to form tablets or fill capsules, or to prepare an aqueous suspension or solution by addition of an aqueous vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Although not a preferred method of administration for a pharmaceutical formulation of oral HDV insulin, as used herein, “parenteral administration” of a pharmaceutical formulation of oral HDV insulin or a pharmaceutical composition comprising oral HDV insulin and one or more therapeutic agents not associated with oral HDV insulin, includes any route of administration characterized by physical breaching of a tissue of a subject and administration of the pharmaceutical composition through the breach in the tissue. Parenteral administration thus includes, but is not limited to, administration application through a surgical incision, by application through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

A pharmaceutical formulation of oral HDV insulin and a pharmaceutical composition comprising oral HDV insulin and one or more therapeutic agents not associated with oral HDV insulin suitable for parenteral administration comprise oral HDV insulin or a pharmaceutical composition comprising oral HDV insulin and one or more therapeutic agents not associated with oral HDV insulin, and one or more additional therapeutic agents combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi dose containers containing a preservative. Formulations for parenteral administration include, but are not limited to, suspensions, emulsions in oily or aqueous vehicles (subject to the stability of oral HDV insulin in an oily vehicle), pastes, and implantable sustained-release or biodegradable formulations. Such formulations may further comprise one or more additional ingredients including, but not limited to, suspending, stabilizing, or dispersing agents.

In one embodiment of a formulation for parenteral administration, oral HDV insulin or a pharmaceutical composition comprising oral HDV insulin and one or more therapeutic agents not associated with oral HDV insulin, and one or more additional therapeutic agents are provided in solid (i.e. powder or granular) form for reconstitution with a suitable vehicle (e.g. sterile pyrogen free water) prior to parenteral administration of the reconstituted pharmaceutical.

The pharmaceutical composition may be prepared, packaged, or sold in the form of a sterile injectable aqueous suspension. This suspension may be formulated according to the known art, and may comprise, in addition to oral HDV insulin or a pharmaceutical composition comprising oral HDV insulin and one or more therapeutic agents not associated with oral HDV insulin, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, and isotonic sodium chloride solution.

Other parentally-administrable formulations which are useful include those which comprise a pharmaceutical formulation of oral HDV insulin or a pharmaceutical composition comprising oral HDV insulin and one or more therapeutic agents not associated with oral HDV insulin in microcrystalline form for ultrasound-released delivery or as a component of a biodegradable polymer system. Pharmaceutical compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) oral HDV insulin or a pharmaceutical composition comprising oral HDV insulin and one or more therapeutic agents not associated with oral HDV insulin, the balance comprising an orally dissolvable or degradable composition and, optionally, one or more of the additional ingredients described herein. Alternately, formulations suitable for buccal administration may comprise a powder or an aerosolized or atomized solution or suspension comprising oral HDV insulin or a pharmaceutical composition comprising oral HDV insulin and one or more therapeutic agents not associated with oral HDV insulin. Such powdered, aerosolized, or aerosolized formulations, when dispersed, preferably have an average particle or droplet size in the range from about 0.1 to about 200 nanometers, and may further comprise one or more of the additional ingredients described herein.

### Kits Including Oral HDV Insulin

The present invention also includes a kit containing a pharmaceutical formulation of oral HDV insulin or a pharmaceutical composition comprising oral HDV insulin and one or more therapeutic agents not associated with oral HDV insulin. Alternatively, a kit may contain a pharmaceutical formulation of oral HDV insulin and one or more therapeutic agents for co-administration with oral HDV insulin.

The above described kits further include instructional material which describes administering the contents of the kit to a mammal. As used herein, an “instructional material” includes a publication, a recording, a diagram, or any other medium of expression which can be used to communicate the usefulness of the composition of the invention in the kit for effecting alleviation of the various diseases or disorders recited herein.

Optionally, or alternatively, the instructional material may describe one or more methods of alleviating the diseases or disorders in a cell or a tissue of a mammal. The instructional material of the kit may, for example, be affixed to a container which contains the invention or be shipped together with a container which contains the invention. Alternatively, the instructional material may be shipped separately from the container with the intention that the instructional material and the compound be used cooperatively by the recipient.

A kit may include a pharmaceutical formulation of oral HDV insulin or a pharmaceutical composition comprising oral HDV insulin and one or more therapeutic agents not associated with oral HDV insulin packaged in a container for convenient QD, BID, TID, qXh (where X is an integer from 1 to 24), weekly, or monthly administration. A kit may further include one or more packages containing sufficient oral HDV insulin or a pharmaceutical composition comprising oral HDV insulin and one or more therapeutic agents not associated with oral HDV insulin for administration for 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or for any and all increments therebetween.

A kit containing a pharmaceutical formulation of oral HDV insulin as described above may further include one or more therapeutic agents for co-administration with oral HDV insulin, packaged in one or more packages either with or separate from oral HDV insulin, in the manner noted above, i.e. for convenient QD, BID, TID, qXh (where X is an integer from 1 to 24), weekly, or monthly administration or with enough one or more therapeutic agents for administration for 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or for any and all increments therebetween.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

### Example 1

### Method of Preparing HDV Insulin

The lipid construct used in the preparation of HDV insulin was prepared by mixing 1,2-distearoyl-sn-glycero-3-phosphocholine, cholesterol, dihexadecyl phosphate, and targeting agent at about 71.2 wt %, 9.4 wt %, 18.2 wt %, and 1.2 wt %, respectively. The mixture of 1,2-distearoyl-sn-glycero-3-phosphocholine, dihexadecyl phosphate, cholesterol, and targeting agent was placed in a 3 liter flask and 45 mls of an anhydrous chloroform/methanol (2:1 v:v) solution was added to the lipid mixture. The solvent was subsequently evaporated under reduced pressure using a rotary evaporator (“rotovap”) with a water bath at about 60° C. The resulting residue was dried under high-vacuum for approximately two hours to remove residual solvent.

Once all organic solvents had been removed, 600 ml of 28.4 mM sodium phosphate (monobasic-dibasic) buffer at pH 7.0 was added to the flask. The flask was then placed in a water bath at about 76 to about 84° C., preferably about 80° C., for 30 minutes while slowly turning to hydrate the lipids.

Next, the hydrated mixture was fed into an M-110 EH microfluidizer, preheated to between about 60 to about 80° C., preferably about 70° C. The suspension of the hydrated lipid construct was transferred to the microfluidizer and microfluidized at approximately 9000 psig using one pass of the suspension of the hydrated construct through the fluidizer. After passing through the microfluidizer, an unfiltered sample (2.0-5.0 ml) of the fluidized suspension was collected for size analysis using unimodal distribution data from a Coulter N-4 plus particle size analyzer. Prior to size determinations, the sample was diluted with 0.2 micron filtered sterile water for injection, USP, that has been pH adjusted to between about 6.5 to about 7.5. The construct was required to range from greater than about 20 nanometers to about 400 nanometers. If the construct size was not within this range, the suspension was passed through the microfluidizer again until the particle size requirements are reached.

Following microfluidization, the resultant suspension was maintained at about 58 to about 62° C., preferably about 60° C., while filtered twice through a sterile 0.8 micron+0.2 micron gang filter.

Insulin is associated with the construct using the “reverse loading” technique described in U.S. Pat. No. 5,104,661. According to this method, 1.0 ml of lipid construct in 39.8 mM NaH₂PO₄—NaOH buffer (pH 7.3) was mixed with 1.0 ml of insulin stock solution prepared at a concentration of 0.874 mM in 15.1 mM NaH₂PO₄—NaOH buffer (pH 7.3). The loading procedure was allowed to proceed for 18 hours at 4° C. Following the incubation in the refrigerator at 4° for 18 hours, HDV was annealed at 45° C. for 20 minutes with slow turning on a rotary evaporator. Post association, the final concentration of insulin in solution was 0.437 mM, and a final buffer concentration of 27.45 mM NaH₂PO₄—NaOH buffer (pH 7.3).

### Example 2

### Method of Preparing Oral HDV Insulin

Generally, the constituents of oral HDV insulin are formed when at least one lipid component and optional targeting agent are homogenized in an aqueous media via microfluidization or other process involving cavitation.

In an embodiment of the invention, the lipid component(s) and optional targeting agent(s) may be homogenized in 18 mM phosphate buffer at a pH of about 6.0 to a pH of about 8.0. Lipid component concentration in the phosphate buffer may range from about 10 to about 200 mg/ml and any and all whole and partial integers therebetween. In one embodiment, the lipid component concentration is about 30 to about 150 mg/ml. In more preferred embodiment, the lipid component concentration is about 15 to about 50 mg/ml. In a most preferred embodiment, the lipid component concentration is about 28-30 mg/ml.

Homogenization of the aqueous media, lipid component(s), and optional targeting agent may be accomplished via treatment in a device suitable for homogenization. Examples of suitable devices include, but are not limited to, a Polytron® System PT 6100, an M-110-EH microfluidizer, an ultrasonic sonicator, a high pressure membrane filtration apparatus, and a homogenizer extruder.

In instances where a microfluidizer is used, the microfluidizer is preferably operated at a temperature that is greater than the highest transition temperature of a lipid component and most preferably at a temperature greater than about 75° C. Thus, the elevated temperature allows any acyl and alkyl chains present in the lipid component(s) to move fluidly as well as conform to and associate with neighboring hydrocarbon moieties. These non-covalent associations directly result in the formation of a constituent of a composition of the present invention.

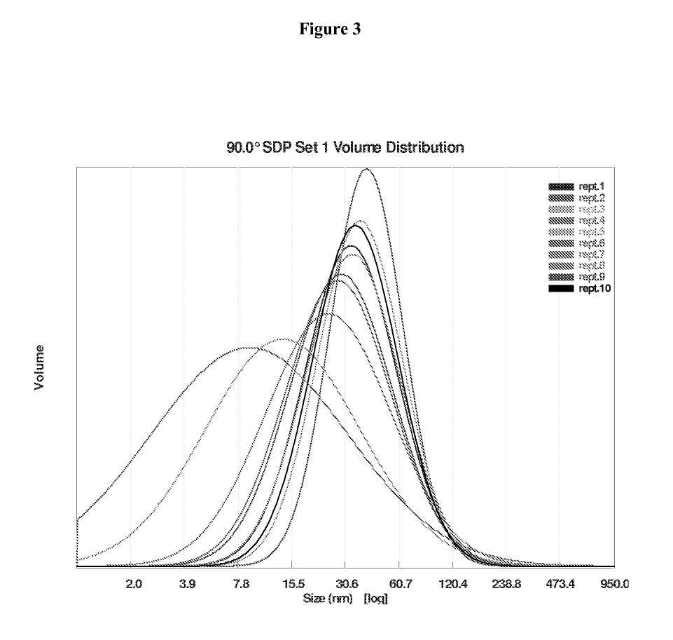

For the microfluidization process, up to about five independent passes are required at 9000 psig in order to achieve dynamic constituent sizing with some constituents possessing radii of less than 20 nanometers. Constituent analysis data generated by a Coulter N-4 Plus Sub-Micron Particle Size Analyzer is shown in FIG. 3 and represents 10 repeated size analyses on the same sample as it remained stationary in the Coulter N-4 Plus Sub-Micron Particle Size Analyzer. This data demonstrates the dynamic nature of constituent sizing and the fluid nature of the interactions between the constituents of oral HDV insulin in aqueous media.

After microfluidization, the resulting constituents may be sterile filtered through a 0.8 micron to 0.2 micron gang Supor™ membrane.

During the process of sub-micron particle formation, hydrogen bonding, ionic bonding, van der Waal's interactions, dipolar interactions, ion-dipole interactions and hydrophobic associations dictate the manner in which the constituents of oral HDV insulin assemble. While not wishing to be bound by any one particular theory, it is believed that the interaction of all of these forces, to varying extents, under the conditions noted above, lead to the dynamically sized constituents of the present invention.

Insulin may be associated with constituents of oral HDV according to the following general procedure. Association is achieved via addition of a low molarity solution of insulin to an aqueous suspension of constituents. In this embodiment, the number of lipid molecules involved in the assembly of the constituents far surpasses the number of molecules of insulin interlaced and/or combined either on or within the constituents' matricies. This high ratio of constituents to insulin minimizes the molecular interactions between insulin and the constituents, insuring that the self-assembly and self-organization processes of the constituents into oral HDV insulin are not disrupted. This high ratio facilitates the formation of a stable constituent/insulin association.

Without wishing to be bound by a particular theory, it is believed that the quantity of insulin associated with oral HDV insulin appears to be a function of loading time and lipid concentration. As the lipid component concentration in aqueous media is increased, additional insulin associates with oral HDV insulin. The time required for loading insulin may be anywhere from several hours to about one week. Preferably, though, insulin is loaded over about 12 to about 18 hours.

The low concentration of insulin relative to the concentration of the constituents of oral HDV insulin is unique among lipid particle delivery systems. Typically, liposome or liposome-like delivery systems have employed a much larger quantity of insulin.

In other embodiments the addition of a higher concentration of insulin may be both desirable and advantageous. The constituent members of oral HDV insulin are capable of associating with, and tolerating, higher molarity solutions of insulin.

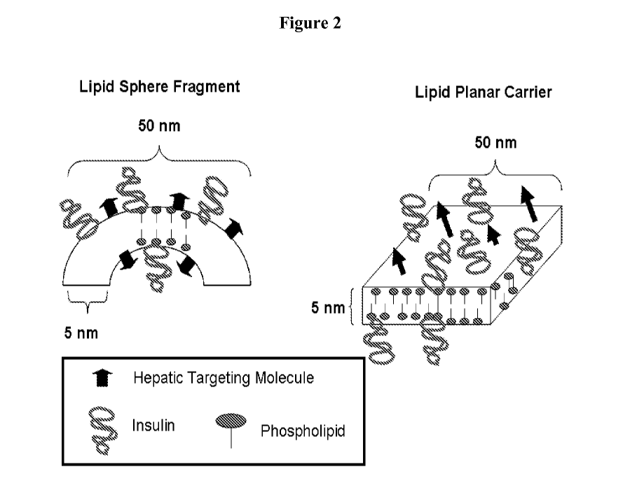

A diagrammatic example insulin associated with oral HDV insulin is depicted in FIG. 2. FIG. 2 illustrates a constituent/HTM/insulin construct. In this diagrammatic representation, insulin is bound to the surface of the constituent via non-covalent electrostatic interactions. Insulin may, however, be otherwise associated with oral HDV insulin, such as via incorporation into a membrane or membrane fragment.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A pharmaceutical composition comprising,
HDV insulin, and
one or more additional therapeutic agents not associated with HDV insulin.

2. The pharmaceutical composition of claim 1, wherein said one or more additional therapeutic agents not associated with HDV insulin are selected from the group consisting of an α-glucosidase inhibitor, a lipase inhibitor, a sulfonyl urea, a meglitinide, a biguanide, a thiazolidinedione, pramlintide, an incretin mimetic, GLP-1 receptor agonist, a DPP-IV inhibitor, asprin, niacin, a fibrate, a bile acid sequestrant, a cholesterol absorption inhibitor, an omega-3 acid ethyl ester, a secretory phospholipase A2 (“sPLA2”) inhibitor, an oligonucleotide-based apolipoprotein B (“apoB”) inhibitor, a squalene synthase inhibitor, a statin, a fixed dose combination statin therapy, glucose, glucagon, heparin, an angiotensin II receptor antagonist, an ACE inhibitor, an antidepressant, an anticonvulsant, an opioid, C-peptide, an aldose reductase inhibitor, a pancreatic lipase inhibitor, a serotonin-norepinephrine reuptake inhibitor, a cannabinoid (“CB1”) receptor antagonist, a leptin receptor agonist, oxyntomodulin or an oxyntomodulin-derived peptide, peptide tyrosine-tyrosine (PYY), an anti-obesity therapy, an anti-obesity combination therapy, an erectile dysfunction medication, alpha-1-adrenergic receptor blockers, 5-alpha reductase inhibitors, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, other non-insulin compounds, and combinations thereof.

3. The pharmaceutical composition of claim 2, wherein
said α-glucosidase inhibitor is selected from the group consisting of acarbose, miglitol, and voglibose;
said lipase inhibitor is orlistat;
said sulfonyl urea is selected from the group consisting of acetohexamide, chlorpropamide, tolbutamide, tolazamide, gliclazide, glyburide, glibenclamide, glipizide glimepiride, and gliquidone;
said meglitinide is selected from mitiglinide, nateglinide, and repaglinide;
said biguanide is selected from the group consisting of metformin, phenformin, and buformin;
said thiazolidinedione is selected from the group consisting of rosiglitazone, pioglitazone, troglitazone, and tesaglitazar;
said incretin mimetic is selected from the group consisting of exenatide and liraglutide;
said DPP-IV inhibitor is selected from the group consisting of sitagliptin, a combination of sitagliptin tin and metformin, vildagliptin, alogliptin, a combination of alogia and metformin, saxagliptin, and a combination of vildagliptin and metformin;
said fibrate is selected from the group consisting of fenofibrate, bezafibrate, and gemfibrozil;
said bile acid sequestrant is selected from the group consisting of colesevelam and cholestyramine;
wherein said cholesterol absorption inhibitor is selected from the group consisting of ezetimibe, FM-VP4, AEGR-733, implitapide and JTT-130;
said omega-3 acid ethyl ester is selected from the group consisting of Omacor™, Esapent™, Seacor™, and Maxepa™;
said secretory phospholipase A2 inhibitor is selected from the group consisting of S-5920, LY315920, and A-002;
said oligonucleotide-based apolipoprotein B inhibitor is mipomersen sodium;
said statin is selected from the group consisting of mevastatin, lovastatin, simvastatin, pravastatin, fluvastatin, pitavastatin, atorvastatin, cerivastatin, and rosuvastatin;
said squalene synthase inhibitor is lapaquisatat;
said fixed dose combination statin therapy is selected from the group consisting of simvastatin and ezetimibe (Vytorin™), atorvastatin and amlodipine (Caduet™), and lovastatin and nicotinic acid (Advicor™);
said angiotensin II receptor antagonist is selected from the group consisting of valsartan, losartan, irbesartan, candesartan celexetil, olmesartan, a combination of losartan and hydrochlorothiazide, and a combination of valsartan and hydrochlorothiazide;
said ACE inhibitor is selected from the group consisting of benazepril, captopril, lisinopril, ramipril, enalapril, a combination of lisinopril and hydrochlorothiazide, and a combination of benazepril and amlodipine;
said antidepressant is selected from the group consisting of amitriptyline, imipramine, desipramine, dapoxetine, venlafaxin, bupropion, paroxetine, citalopram, dapoxetine, escitalopram, fluoxetine, fluvoxamine, paroxetine, sertraline, and zimelidine;
said anticonvulsant is selected from the group consisting of pregabalin, gabapentin, carbamazepine, lamotrigine, and topiramate;
said opioid is selected from morphine, codeine, thebaine, hydromorphone, hydrocodone, oxycodone, oxymorphone, desomorphine, nicomorphine, dipropanoylmorphine, benzylmorphine, ethylmorphine, fentanyl, pethidine, methadone, tramadol and propoxyphene;
said aldose reductase inhibitor is selected from epalrestat and ranirestat;
said pancreatic lipase inhibitor is orlistat;
said serotonin-norepinephrine reuptake inhibitor is sibutramine;
said cannabinoid receptor antagonist is selected from the group consisting of rimonabant and MK-0364;
said anti-obesity combination therapy is selected from the group consisting of a combination of topiramate and phentermine, a combination of bupropion and zonisamide, a combination of bupropion and naltrexone, a combination of phentermine and fluoxetine, a combination of phentermine and sertraline, a combination of phentermine and citalopram, a combination of phentermine and escitalopram, and a combination of phentermine and trazadone;
said erectile dysfunction medication is selected from the group consisting of alprostadil, tadalafil, vardenafil, and sildenafil,
said fish oil is selected from the group consisting of eicosapentaenoic acid “EPA” and docosahexaenoic acid (“DHA”) and combinations thereof; and,
said plant sterols and stanols are selected from the group consisting of β-sitosterol, β-sitostanol, campesterol, sigmasterol and combinations thereof.

4-32. (canceled)

33. A pharmaceutical composition comprising,
oral HDV insulin, and
one or more additional therapeutic agents not associated with oral HDV insulin.

34. The pharmaceutical composition of claim 33, wherein said one or more additional therapeutic agents not associated with oral HDV insulin are selected from the group consisting of an α-glucosidase inhibitor, a lipase inhibitor, a sulfonyl urea, a meglitinide, a biguanide, a thiazolidinedione, pramlintide, an incretin mimetic, GLP-1 receptor agonist, a DPP-IV inhibitor, asprin, niacin, a fibrate, a bile acid sequestrant, a cholesterol absorption inhibitor, an omega-3 acid ethyl ester, a secretory phospholipase A2 (“sPLA2”) inhibitor, an oligonucleotide-based apolipoprotein B (“apoB”) inhibitor, a squalene synthase inhibitor, a statin, a fixed dose combination statin therapy, glucose, glucagon, heparin, an angiotensin II receptor antagonist, an ACE inhibitor, an antidepressant, an anticonvulsant, an opioid, C-peptide, an aldose reductase inhibitor, a pancreatic lipase inhibitor, a serotonin-norepinephrine reuptake inhibitor, a cannabinoid (“CB1”) receptor antagonist, a leptin receptor agonist, oxyntomodulin or an oxyntomodulin-derived peptide, peptide tyrosine-tyrosine (PYY), an anti-obesity therapy, an anti-obesity combination therapy, an erectile dysfunction medication, an alpha-1-adrenergic receptor blocker, a 5-alpha reductase inhibitor, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, other non-insulin compounds, and combinations thereof.

35. The pharmaceutical composition of claim 34, wherein
said α-glucosidase inhibitor is selected from the group consisting of acarbose, miglitol, and voglibose;
said lipase inhibitor is orlistat;
said sulfonyl urea is selected from the group consisting of acetohexamide, chlorpropamide, tolbutamide, tolazamide, gliclazide, glyburide, glibenclamide, glipizide, glimepiride, and gliquidone;
said meglitinide is selected from mitiglinide, nateglinide, and repaglinide;
said biguanide is selected from the group consisting of metformin, phenformin, and buformin;
said thiazolidinedione is selected from the group consisting of rosiglitazone, pioglitazone, troglitazone, and tesaglitazar;
said incretin mimetic is selected from the group consisting of exenatide and liraglutide;
said DPP-IV inhibitor is selected from the group consisting of sitagliptin, a combination of sitagliptin and metformin, vildagliptin, alogliptin, a combination of alogliptin and metformin, saxagliptin, and a combination of vildagliptin and metformin;
said fibrate is selected from the group consisting of fenofibrate, bezafibrate, and gemfibrozil;
said bile acid sequestrant is selected from the group consisting of colesevelam and cholestyramine;
wherein said cholesterol absorption inhibitor is selected from the group consisting of ezetimibe, FM-VP4, AEGR-733, implitapide and JTT-130;
said omega-3 acid ethyl ester is selected from the group consisting of Omacor™, Esapent™, Seacor™, and Maxepa™;
said secretory phospholipase A2 inhibitor is selected from the group consisting of S-5920, LY315920, and A-002;
said oligonucleotide-based apolipoprotein B inhibitor is mipomersen sodium;
said statin is selected from the group consisting of mevastatin, lovastatin, simvastatin, pravastatin, fluvastatin, pitavastatin, atorvastatin, cerivastatin, and rosuvastatin;
said squalene synthase inhibitor is lapaquisatat;
said fixed dose combination stalin therapy is selected from the group consisting of simvastatin and ezetimibe (Vytorin™), atorvastatin and amlodipine (Caduet™), and lovastatin and nicotinic acid (Advicor™);
said angiotensin II receptor antagonist is selected from the group consisting of valsartan, losartan, irbesartan, candesartan celexetil, olmesartan, a combination of losartan and hydrochlorothiazide, and a combination of valsartan and hydrochlorothiazide;
said ACE inhibitor is selected from the group consisting of benazepril, captopril, lisinopril, ramipril, enalapril, a combination of lisinopril and hydrochlorothiazide, and a combination of benazepril and amlodipine;
said antidepressant is selected from the group consisting of amitriptyline, imipramine, desipramine, duloxetine, venlafaxin, bupropion, paroxetine, citalopram, dapoxetine, escitalopram, fluoxetine, fluvoxamine, paroxetine, sertraline, and zimelidine;
said anticonvulsant is selected from the group consisting of pregabalin, gabapentin, carbamazepine, lamotrigine, and topiramate;
said opioid is selected from morphine, codeine, thebaine, hydromorphone, hydrocodone, oxycodone, oxymorphone, desomorphine, nicomorphine, dipropanoylmorphine, benzylmorphine, ethylmorphine, fentanyl, pethidine, methadone, tramadol and propoxyphene;
said aldose reductase inhibitor is selected from epairestat and ranirestat;
said pancreatic lipase inhibitor is orlistat;
said serotonin-norepinephrine reuptake inhibitor is sibutramine;
said cannabinoid receptor antagonist is selected from the group consisting of rimonabant and MK-0364;
said anti-obesity combination therapy is selected from the group consisting of a combination of topiramate and phentermine, a combination of bupropion and zonisamide, a combination of bupropion and naltrexone, a combination of phentermine and fluoxetine, a combination of phentermine and sertraline, a combination of phentermine and citalopram, a combination of phentermine and escitalopram, and a combination of phentermine and trazadone;
said erectile dysfunction medication is selected from the group consisting of alprostadil, tadalafil, vardenafil, and sildenafil;
said fish oil is selected from the group consisting of eicosapentaenoic acid (“EPA”) and docosahexaenoic acid (“DHA”) and combinations thereof; and
said plant sterols and stanols are selected from the group consisting of β-sitosterol, β-sitostanol, campesterol, sigmasterol and combinations thereof.

36-64. (canceled)

65. A method of preparing a pharmaceutical composition comprising HDV insulin and one or more additional therapeutic agents not associated with HDV insulin, said method comprising the steps of:
a. mixing lipid components and at least one targeting agent in aqueous media to form a first mixture;
b. adding at least one insulin to said first mixture to form a second mixture; and
c. formulating said second mixture with said one or more additional therapeutic agents.

66. A method of preparing a pharmaceutical composition comprising oral HDV insulin and one or more additional therapeutic agents not associated with oral HDV insulin, said method comprising the steps of:
a. forming oral HDV insulin by:
i. mixing lipid components and, optionally, at least one targeting agent in aqueous media to form a first mixture;
ii. adding at least one insulin to said first mixture to form a second mixture;
iii. adding said second mixture to gelatin to form a gelatin-associated mixture;
iv. drying said gelatin-associated mixture to form a dry mixture of oral HDV insulin; and
b. formulating said dry mixture of oral HDV insulin with said one or more additional therapeutic agents not associated with oral HDV insulin.

67. The method of claim 65, wherein said one or more therapeutic agents are selected from the group consisting of an α-glucosidase inhibitor, a lipase inhibitor, a sulfonyl urea, a meglitinide, a biguanide, a thiazolidinedione, pramlintide, an incretin mimetic, GLP-1 receptor agonist, a DPP-IV inhibitor, asprin, niacin, a fibrate, a bile acid sequestrant, a cholesterol absorption inhibitor, an omega-3 acid ethyl ester, a secretory phospholipase A2 (“sPLA2”) inhibitor, an oligonucleotide-based apolipoprotein B (“apoB”) inhibitor, a squalene synthase inhibitor, a statin, a fixed dose combination statin therapy, glucose, glucagon, heparin, an angiotensin II receptor antagonist, an ACE inhibitor, an antidepressant, an anticonvulsant, an opioid, C-peptide, an aldose reductase inhibitor, a pancreatic lipase inhibitor, a serotonin-norepinephrine reuptake inhibitor, a cannabinoid (“CB1”) receptor antagonist, a leptin receptor agonist, oxyntomodulin or an oxyntomodulin-derived peptide, peptide tyrosine-tyrosine (PYY), an anti-obesity therapy, an anti-obesity combination therapy, an erectile dysfunction medication, an alpha-1-adrenergic receptor blocker, a 5-alpha reductase inhibitor, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, other non-insulin compounds, and combinations thereof.

68. The method of claim 67, wherein said
α-glucosidase inhibitor is selected from the group consisting of acarbose, and voglibose;
said lipase inhibitor is orlistat;
said sulfonyl urea is selected from the group consisting of acetohexamide, chlorpropamide, tolbutamide, tolazamide, gliclazide, glyburide, glibenclamide, glipizide, glimepiride, and gliquidone;
said meglitinide is selected from mitiglinide, nateglinide, and repaglinide;
said biguanide is selected from the group consisting of metformin, phenformin, and buformin;
said thiazolidinedione is selected from the group consisting of rosiglitazone, pioglitazone, troglitazone, and tesaglitazar;
said incretin mimetic is selected from the group consisting of exenatide and liraglutide;
said DPP-IV inhibitor is selected from the group consisting of sitagliptin, a combination of sitagliptin and metformin, vildagliptin, alogliptin, a combination of alogliptin and metformin, saxagliptin, and a combination of vildagliptin and metformin;
said fibrate is selected from the group consisting of fenofibrate, bezafibrate, and gemfibrozil;
said bile acid sequestrant is selected from the group consisting of colesevelam and cholestyramine;
wherein said cholesterol absorption inhibitor is selected from the group consisting of ezetimibe, FM-VP4, AEGR-733, implitapide and JTT-130;
said omega-3 acid ethyl ester is selected from the group consisting of Omacor™, Esapent™, Seacor™, and Maxepa™;
said secretory phospholipase A2 inhibitor is selected from the group consisting of S-5920, LY315920, and A-002;
said oligonucleotide-based apolipoprotein B inhibitor is mipomersen sodium;
said statin is selected from the group consisting of mevastatin, lovastatin, simvastatin, pravastatin, fluvastatin, pitavastatin, atorvastatin, cerivastatin, and rosuvastatin;
said squalene synthase inhibitor is lapaquisatat;
said fixed dose combination statin therapy is selected from the group consisting of simvastatin and ezetimibe (Vytorin™), atorvastatin and amlodipine (Caduet™), and lovastatin and nicotinic acid (Advicor™);
said angiotensin II receptor antagonist is selected from the group consisting of valsartan, losartan, irbesartan, candesartan celexetil, olmesartan, a combination of losartan and hydrochlorothiazide, and a combination of valsartan and hydrochlorothiazide;
said ACE inhibitor is selected from the group consisting of benazepril, captopril, lisinopril, ramipril, enalapril, a combination of lisinopril and hydrochlorothiazide, and a combination of benazepril and amlodipine;
said antidepressant is selected from the group consisting of amitriptyline, imipramine, desipramine, duloxetine, venlafaxin, bupropion, paroxetine, citalopram, dapoxetine, escitalopram, fluoxetine, fluvoxamine, paroxetine, sertraline, and zimelidine;
said anticonvulsant is selected from the group consisting of pregabalin, gabapentin, carbamazepine, lamotrigine, and topiramate;
said opioid is selected from morphine, codeine, thebaine, hydromorphone, hydrocodone, oxycodone, oxymorphone, desomorphine, nicomorphine, dipropanoylmorphine, benzylmorphine, ethylmorphine, fentanyl, pethidine, methadone, tramadol and propoxyphene;
said aldose reductase inhibitor is selected from epalrestat and ranirestat;
said pancreatic lipase inhibitor is orlistat;
said serotonin-norepinephrine reuptake inhibitor is sibutramine;
said cannabinoid receptor antagonist is selected from the group consisting of rimonabant and MK-0364;
said anti-obesity combination therapy is selected from the group consisting of a combination of topiramate and phentermine, a combination of bupropion and zonisamide, a combination of bupropion and naltrexone, a combination of phentermine and fluoxetine, a combination of phentermine and sertraline, a combination of phentermine and citalopram, a combination of phentermine and escitalopram, and a combination of phentermine and trazadone;
said erectile dysfunction medication is selected from the group consisting of alprostadil, tadalafll, vardenafil, and sildenafil;
said fish oil is selected from the group consisting of eicosapentaenoic acid (“EPA”) and docosahexaenoic acid (“DHA”) and combinations thereof; and
said plant sterols and stanols are selected from the group consisting of β-sitosterol, β-sitostanol, campesterol, sigmasterol and combinations thereof.

69-97. (canceled)

98. The method of claim 66, wherein said one or more therapeutic agents are selected from the group consisting of an α-glucosidase inhibitor, a lipase inhibitor, a sulfonyl urea, a meglitinide, a biguanide, a thiazolidinedione, pramlintide, an incretin mimetic, GLP-1 receptor agonist, a DPP-IV inhibitor, asprin, niacin, a fibrate, a bile acid sequestrant, a cholesterol absorption inhibitor, an omega-3 acid ethyl ester, a secretory phospholipase A2 (“sPLA2”) inhibitor, an oligonucleotide-based apolipoprotein B (“apoB”) inhibitor, a squalene synthase inhibitor, a statin, a fixed dose combination statin therapy, glucose, glucagon, heparin, an angiotensin II receptor antagonist, an ACE inhibitor, an antidepressant, an anticonvulsant, an opioid, C-peptide, an aldose reductase inhibitor, a pancreatic lipase inhibitor, a serotonin-norepinephrine reuptake inhibitor, a cannabinoid (“CB1”) receptor antagonist, a leptin receptor agonist, oxyntomodulin or an oxyntomodulin-derived peptide, peptide tyrosine-tyrosine (PYY), an anti-obesity therapy, an anti-obesity combination therapy, an erectile dysfunction medication, an alpha-1-adrenergic receptor blocker, a 5-alpha reductase inhibitor, fish oil, plant sterols and stanols, immunosuppressors, SGLT2 inhibitors, 11βSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, other non-insulin compounds, and combinations thereof.

99. The method of claim 98, wherein
said α-glucosidase inhibitor is selected from the group consisting of acarbose, miglitol, and voglibose;
said lipase inhibitor is orlistat;
said sulfonyl urea is selected from the group consisting of acetohexamide, chlorpropamide, tolbutamide, tolazamide, gliclazide, glyburide, glibenclamide, glipizide, glimepiride, and gliquidone;
said meglitinide is selected from mitiglinide, nateglinide, and repaglinide;
said biguanide is selected from the group consisting of metformin, phenformin, and buformin;
said thiazolidinedione is selected from the group consisting of rosiglitazone, pioglitazone, troglitazone, and tesaglitazar;
said incretin mimetic is selected from the group consisting of exenatide and liraglutide;
said DPP-IV inhibitor is selected from the group consisting of sitagliptin, a combination of sitagliptin and metformin, vildagliptin, alogliptin, a combination of alogliptin and metformin, saxagliptin, and a combination of vildagliptin and metformin;
said fibrate is selected from the group consisting of fenofibrate, bezafibrate, and gemfibrozil;
said bile acid sequestrant is selected from the group consisting of colesevelam and cholestyramine;
wherein said cholesterol absorption inhibitor is selected from the group consisting of ezetimibe, FM-VP4, AEGR-733, implitapide and JTT-130
said omega-3 acid ethyl ester is selected from the group consisting of Omacor™, Esapent™, Seacor™ and Maxepa™;
said secretory phospholipase A2 inhibitor is selected from the group consisting of S-5920, LY315920, and A-002;
said oligonucleotide-based apolipoprotein B inhibitor is mipomersen sodium;
said statin is selected from the group consisting of mevastatin, lovastatin, simvastatin, pravastatin, fluvastatin, pitavastatin, atorvastatin, cerivastatin, and rosuvastatin;
said squalene synthase inhibitor is lapaquisatat;
said fixed dose combination statin therapy is selected from the group consisting of simvastatin and ezetimibe (Vytorin™), atorvastatin and amlodipine (Caduet™), and lovastatin and nicotinic acid (Advicor™);
said angiotensin II receptor antagonist is selected from the group consisting of valsartan, losartan, irbesartan, candesartan celexetil, olmesartan, a combination of losartan and hydrochlorothiazide, and a combination of valsartan and hydrochlorothiazide;
said ACE inhibitor is selected from the group consisting of benazepril, captopril lisinopril, ramipril, enalapril, a combination of lisinopril and hydrochlorothiazide, and a combination of benazepril and amlodipine;
said antidepressant is selected from the group consisting of amitriptyline, imipramine, desipramine, duloxetine, venlafaxin, bupropion, paroxetine, citalopram, dapoxetine, escitalopram, fluoxetine, fluvoxamine, paroxetine, sertraline, and zimelidine;
said anticonvulsant is selected from the group consisting of pregabalin, gabapentin, carbamazepine, lamotrigine, and topiramate;
said opioid is selected from morphine, codeine, thebaine, hydromorphone, hydrocodone, oxycodone, oxymorphone, desomorphine, nicomorphine, dipropanoylmorphine, benzylmorphine, ethylmorphine, fentanyl, pethidine, methadone, tramadol and propoxyphene;
said aldose reductase inhibitor is selected from epalrestat and ranirestat;
said pancreatic lipase inhibitor is orlistat;
said serotonin-norepinephrine reuptake inhibitor is sibutramine;
said cannabinoid receptor antagonist is selected from the group consisting of rimonabant and MK-0364;
said anti-obesity combination therapy is selected from the group consisting of a combination of topiramate and phentermine, a combination of bupropion and zonisamide, a combination of bupropion and naltrexone, a combination of phentermine and fluoxetine, a combination of phentermine and sertraline, a combination of phentermine and citalopram, a combination of phentermine and escitalopram, and a combination of phentermine and trazadone;
said erectile dysfunction medication is selected from the group consisting of alprostadil, tadalafil, vardenafil, and sildenafil;
said fish oil is selected from the group consisting of eicosapentaenoic acid (“EPA”) and docosahexaenoic acid (“DHA”) and combinations thereof; and
said plant sterols and stanols are selected from the group consisting of β-sitosterol, β-sitostanol, campesterol, sigmasterol and combinations thereof.

100-128. (canceled)

129. A method of treating diabetes, a diabetes related ailment, and/or a disease or condition other than diabetes in a patient in need thereof, said method comprising administering to said patient a composition selected from the group consisting of the pharmaceutical composition of claim 1 and the pharmaceutical composition of claim 33.

130. (canceled)

131. A method of treating a diabetes related ailment and/or a disease or conditions other than diabetes or a diabetes related ailment in a patient in need thereof, said method comprising
administering to said patient a pharmaceutical formulation of HDV insulin or pharmaceutical formulation of oral HDV insulin, and
optionally co-administering to said patient one or more additional therapeutic agents not associated with said HDV insulin or said oral HDV insulin.

132. The method of claim 131, wherein said diabetes related ailment and disease or condition other than diabetes is selected from the group consisting of, obesity, fatty liver, cardiovascular disease, diabetic coma, diabetic nephrophathy, diabetic neuropathy, erectile dysfunction, metabolic syndrome, diabetic retinopathy, peripheral insulin level elevation, pre-diabetes, cerebral vasospasm, coronary vasospasm, bronchial asthma, preterm labor, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, or benign prostatic hyperplasia, peripheral vascular disease, gout, dementia, and loss of mental acuity.

133-134. (canceled)

135. The method according to claim 131 wherein said one or more additional therapeutic agent is selected from the group consisting of an α-glucosidase inhibitor, a lipase inhibitor, a sulfonyl urea, a meglitinide, a biguanide, a thiazolidinedione, pramlintide, an incretin mimetic, GLP-1 receptor agonist, a DPP-IV inhibitor, aspirin, niacin, a fibrate, a bile acid sequestrant, a cholesterol absorption inhibitor, an omega-3 acid ethyl ester, a secretory phospholipase A2 (“sPLA2”) inhibitor, an oligonucleotide-based apolipoprotein B (“apoB”) inhibitor, a squalene synthase inhibitor, a statin, a fixed dose combination statin therapy, glucose, glucagon, heparin, an angiotensin II receptor antagonist, an ACE inhibitor, an antidepressant, an anticonvulsant, an opioid, C-peptide, an aldose reductase inhibitor, a pancreatic lipase inhibitor, a serotonin-norepinephrine reuptake inhibitor, a cannabinoid (“CB1”) receptor antagonist, a leptin receptor agonist, oxyntomodulin or an oxyntomodulin-derived peptide, peptide tyrosine-tyrosine (PYY), an anti-obesity therapy, an anti-obesity combination therapy, an erectile dysfunction medication, an alpha-1-adrenergic receptor blocker, a 5-alpha reductase inhibitor, fish oil, plant sterols and stanols, immunosuppressors, SGLT2 inhibitors, 11βHSD1 inhibitors, adenosine A1 receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, MAbs to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, other non-insulin compounds, and combinations thereof.

136. A kit comprising
a. a pharmaceutical composition comprising an insulin selected from the group consisting of HDV insulin and oral HDV insulin, and one or more additional therapeutic agents not associated with said insulin; and
b. instructional material for administration of said composition to a human.

137. (canceled)

138. A kit comprising
a. a pharmaceutical formulation of an insulin selected from the group consisting of HDV insulin and oral HDV insulin, and one or more additional therapeutic agents for co-administration; and
b. instructional material for co-administration of said pharmaceutical formulation of insulin and said therapeutic agents to a human.

139. (canceled)
